(19) United States
(12) Patent Application Publication
(10) Pub. No.: US 2024/0350162 A1
(43) Pub. Date: Oct. 24, 2024

(54) ULTRASONIC TRANSDUCER

(71) Applicant: THE UNIVERSITY COURT OF THE UNIVERSITY OF GLASGOW, Glasgow, Strathclyde (GB)

(72) Inventors: Nicola Giuseppe FENU, Paisley (GB); Sandy COCHRAN, Glasgow (GB); Margaret LUCAS, Glasgow (GB); Rupert SHINGLETON, Oxford (GB); Rebecca Shirley CLEARY, Paisley, Renfrewshire (GB)

(21) Appl. No.: 18/292,766

(22) PCT Filed: Jul. 29, 2022
(86) PCT No.: PCT/EP2022/071469

§ 371 Date: Jan. 26, 2024

(30) Foreign Application Priority Data
Jul. 29, 2021 (GB) 2110897.2

(51) Int. Cl. A61B 17/32; A61B 17/00

(52) U.S. Cl. CPC A61B 17/320068; A61B 2017/00137; A61B 2017/320074

(57) ABSTRACT

An ultrasonic transducer for surgical applications is disclosed. The ultrasonic transducer comprises: a back mass; a front mass; an ultrasonic actuator arrangement held between the back mass and the front mass; and an ultrasonic horn arrangement forward of the front mass. The back mass, ultrasonic actuator arrangement, front mass and ultrasonic horn arrangement are arranged along a longitudinal axis of the transducer. Vibrations generated by the ultrasonic actuator arrangement are conducted into the front mass and into the ultrasonic horn arrangement along a vibrational energy transfer path and are amplitude amplified by the ultrasonic horn arrangement. One or more of the back mass, front mass and ultrasonic horn arrangement includes a plurality of openings opening towards the longitudinal axis and intersecting the vibrational energy transfer path and configured to provide an increased mechanical compliance in a direction along the vibrational energy transfer path.

This application claims priority from GB2110897.2 filed 29 Jul. 2021, the contents and elements of which are herein incorporated by reference for all purposes.

### FIELD OF THE INVENTION

The present invention relates to an ultrasonic transducer and to a method of operation of an ultrasonic transducer. Such an ultrasonic transducer is of particular, although not necessarily exclusive, interest for surgical applications.

### BACKGROUND

The designs of modern ultrasonic surgical devices either for hard or soft tissue almost always resemble the configuration invented by Paul Langevin and Chilowsky in 1922 for underwater applications [1]. Generally, these devices adopt ultrasonic vibrations to enhance cutting performance, employing a transducer mounted in a hand-held device. In the case of soft tissue cutting some common features can be identified. FIG. 1 shows the Harmonic® ACE®+Shears (Ethicon® Endosurgery, Johnson & Johnson, Cincinnati, Ohio, USA), where the waveguide 10, handpiece 12 and Langevin transducer 14 can be recognised. The inset in FIG. 1 also shows the vibration direction “Vib”, longitudinal in this case (parallel to the longitudinal axis of the transducer and waveguide), and the jaw-blade mechanism comprising a pivotable jaw 16 and the vibrating blade 18.

The Harmonic® ACE®+Shears together with the SENHANCE™ Ultrasonic (BOWA® Medical, BOWA-electronic Gmbh & Co. KG, Gomaringen, Germany) represent the only two ultrasonic cutting devices compatible with robotic surgery platforms at the time of writing, those platforms, respectively, the Da Vinci® Surgical System (Intuitive® Surgical Inc., Sunnyvale, California, USA) and the SENHANCE™ Surgical System (BOWA® Medical, Gomaringen, Germany).

One of the most recent inventions for Da Vinci® energy instruments is the EndoWrist® (Intuitive® Surgical Inc.). This articulated joint enables a range of motions at the end effector similar to that of the human wrist, thus replicating the experience of open surgery. FIG. 2 shows a generic Da Vinci® end-effector with EndoWrist® technology. The end-effector 20 is attached at the end of shaft 22 and has first pivot joint 24 permitting flexion and extension (rotation about a first axis) and a second pivot joint 26 permitting adduction and abduction (rotation about a second axis, orthogonal (in this case) with respect to the first axis). A grasping tool 28 is disposed at the distal end of the end-effector.

Several energy instruments are available at the time of writing with EndoWrist® technology, of 5-10 mm in diameter, which allows them to be inserted through a laparoscopic port and maneuvered inside the human body. To date, an ultrasonic cutting device with EndoWrist® technology does not exist. FIG. 2 shows the range of motions available for an EndoWrist® generic end-effector [2].

In current ultrasonic cutting devices, only axial and rotation movements are permitted, as the transducers are too large to fit through the 5-10 mm trocar. The transducer is axially constrained to the robotic arm, and the waveguide, which transfers the ultrasonic vibration from outside the human body to the end effector inside, cannot be bent. This represents a significant disadvantage for surgical ultrasonic devices. In fact, although ultrasonic cutting has shown to be more precise and effective than other energy instruments, and with excellent coagulation speed, the latter are still preferred due to their dexterity.

U.S. Pat. No. 9,408,622 B2 discloses a bendable waveguide that addresses some of the problems identified above but still provides only limited dexterity.

Ultrasonically-actuated surgical tools are an alternative to electrocautery tools, with the potential to avoid some of the disadvantages of electrocautery tools, whilst achieving similar outcomes.

The Harmonic® ACE+Shears is the only ultrasonic device compatible with the Da Vinci® Surgical System. One of its main drawbacks is the lack of flexibility, as it is not compatible with EndoWrist®. The main reason for this is the size of the ultrasonic transducer which is too long and large to be clamped at the end of an end-effector wristed joint to fit through a burr hole or laparoscopic port.

Long rigid waveguides are preferred to transfer the vibrational energy at ultrasonic frequencies from outside to inside the human body. This, inevitably, limits ultrasonic energy instruments to a few applications not requiring accessibility to difficult sites.

Table 1 outlines the advantages and disadvantages of ultrasonic cutting instruments compared with monopolar and bipolar surgical tools for minimally invasive surgery. Despite the numerous advantages reported in Table 5.1, the lack of flexibility of the device in terms of maneuverability limits its application in several procedures as only axial targets can be reached, due to the long straight waveguide and the lack of articulated joints at the blade tip.

TABLE 1
Advantages and disadvantages of ultrasonic energy
instruments, compared with monopolar and bipolar elctrocautery
surgical tools for minimally invasive surgery [6], [7].

| Advantages of ultrasonic | Disadvantages of ultrasonic |
|---|---|
| surgical tools for soft tissue | surgical tools for soft tissue |
| Decreased thermal spread. | Smoke formation while cutting |
| Reduced coagulum. | Lack of flexibility, not |
| Minimal collateral damage | compatible with EndoWrist ® |
| Multifunctionality of the instrument. | Surrounding damage when used in |
| Cut and seal vessels up to 7 mm. | reduced space, difficult to |
| Less tissue sticking to the instrument. | manoeuvre. |
| | Temperature of the shaft (>190° C.). |

Ultrasonic technology is adopted in robotic abdominal laparoscopic surgery for parenchymal (functional tissue) transections where the parenchyma of an organ is dissected from connective and supporting tissue. Ultrasonic energy is also used in lobectomy procedures for the removal of a part of an organ. Other common laparoscopic procedures where ultrasonic energy is preferred, despite the unarticulated instrument, include gastrectomy, adrenalectomy, splenectomy and hepatectomy [6].

The present invention has been devised in light of the above considerations.

### SUMMARY OF THE INVENTION

With current state-of the-art technology of Langevin transducer design, miniaturisation of an ultrasonic surgical tool such as a dissector cannot be achieved while still preserving device functionality and performance.

Accordingly, the present inventors have addressed the issues identified above by considering in detail the effect of the mechanical compliance of the components of a transducer along the vibrational energy transfer path of the transducer.

In this disclosure, we present different “developments” of the present invention, each comprising different optional aspects and further optional features. These are presented below as Development A and Development B.

### Development A

In a first aspect of Development A, the present invention provides an ultrasonic transducer for surgical applications, the ultrasonic transducer comprising:
a back mass;
a front mass;
an ultrasonic actuator arrangement held between the back mass and the front mass;
an ultrasonic horn arrangement forward of the front mass,
wherein vibrations generated by the ultrasonic actuator arrangement are conducted into the front mass and into the ultrasonic horn arrangement along a vibrational energy transfer path and are amplitude amplified by the ultrasonic horn arrangement,
and wherein one or more of the back mass, front mass and ultrasonic horn arrangement includes a plurality of openings intersecting the vibrational energy transfer path and configured to provide an increased mechanical compliance in a direction along the vibrational energy transfer path.

In a second aspect of Development A, the present invention provides a surgical tool comprising an ultrasonic transducer according to the first aspect.

In a third aspect of Development A, the present invention provides a method of operation of an ultrasonic transducer, the ultrasonic transducer comprising:
a back mass;
a front mass;
an ultrasonic actuator arrangement held between the back mass and the front mass;
an ultrasonic horn arrangement forward of the front mass, the method including applying an electrical signal to the ultrasonic actuator arrangement to generate vibrations to be conducted into the front mass and into the ultrasonic horn arrangement along a vibrational energy transfer path and amplitude amplified by the ultrasonic horn arrangement,
wherein one or more of the back mass, front mass and ultrasonic horn arrangement includes a plurality of openings intersecting the vibrational energy transfer path providing an increased mechanical compliance in a direction along the vibrational energy transfer path.

In a fourth aspect of Development A, the present invention provides a method of cutting tissue by ultrasonic cutting using an ultrasonic blade, including a method of operating of an ultrasonic transducer as set out in the third aspect and conducting the vibrational energy to the ultrasonic blade.

The transducer may be a Langevin type transducer, such as a bolted Langevin transducer. Such transducers are of interest for surgical applications due to their low frequency and high power operating characteristics.

The ultrasonic actuator arrangement may comprise a piezoelectric material element such as a piezoceramic element. A plurality of such elements may be provided. One or more associated electrodes may be provided in order to conduct a driving signal to the piezoelectric material element(s). Where the front and/or back mass is formed of an electrically conductive material such as metal, the front and/or back mass may provide ground electrical contact(s) for the piezoelectric material element(s).

The vibrational energy transfer path in the back mass, front mass and/or ultrasonic horn may include an annular portion. The annular portion may take the form of a hollow cylinder for example, with the wall of the cylinder extending along the vibrational energy transfer path and, in operation, vibrational energy passing along the wall of the cylinder. The plurality of openings intersecting the vibrational energy transfer path may therefore be provided through the wall of the annular portion. The openings are typically through-holes. Although it is possible in principle to use blind holes, the subsequent operation of the transducer is expected not to be as suitable.

Each opening may have a substantially uniform cross section along its depth. Considering the depth direction of the opening as the direction parallel to the walls of the opening, the depth direction may be substantially perpendicular to the vibrational energy transfer path (e.g. locally in the transducer).

The openings may each have the same size (e.g. the same diameter if the openings have circular cross sectional shape, or other characteristic linear dimension(s) for other shapes). The openings may have the same cross sectional area. Furthermore, the openings may have the same shape as each other, or may be selected from a limited number of shapes (e.g. two, three or four).

The openings may be arranged based on a repeating pattern. For example the openings may be arranged based on a regular lattice such as a square lattice, rectangular lattice, triangular lattice, hexagonal lattice.

Alternatively the openings may be arranged substantially randomly, or offset randomly from a notional regular repeating pattern.

There may be three or more openings. For example there may be 5 or more, 10 or more, 15 or more, 20 or more, 25 or more, 30 or more, 35 or more, 40 or more, 45 or more or 50 or more openings. There are typically fewer than 500 openings, although in some embodiments there may be more openings.

Suitable cross sectional shapes for the openings include circular, oval, elliptical, round, triangular, quadrilateral, rectangular, square, rhombus, pentagonal, hexagonal, pentagonal, octagonal etc. A combination of two or more such shapes may be used for the openings. The openings may have randomly-generated shape. The openings typically have a closed perimeter.

The openings may have a cross sectional area of, for example, at least 0.01 mm², at least 0.05 mm², at least 0.1 mm², at least 0.2 mm², at least 0.4 mm², at least 0.6 mm², at least 0.8 mm², at least 1 mm², at least 1.5 mm², or at least 2 mm². There is no particular upper limit on the cross sectional area of the openings, except that a suitable number of openings will need to be fitted to the overall size of the transducer in order to have an effect on the compliance of the relevant component.

The openings may be formed by any suitable manufacturing technique, e.g. machining, cutting (e.g. laser cutting) or etching, or by manufacturing the component with net shape or near net shape techniques such as casting, additive manufacture, etc.

The openings may be filled with a filler material. The filler material may have a Young's modulus that is lower (e.g. a factor of at least 5 lower, at least 10 lower, at least 20 lower or at least 50 lower) than the Young's modulus of the material through with the openings are formed. Suitable materials include epoxy materials or other resin-based materials.

Openings may be formed in two or more of the back mass, front mass and ultrasonic horn arrangement.

The operating frequency of the transducer may for example be at least 1 kHz, at least 5 kHz, at least 10 kHz, at least 20 kHz, or at least 30 kHz or at least 40 KHz. The operating frequency of the transducer may for example be at most 200 kHz, at most 150 kHz, at most 100 kHz, or at most 90 kHz, at most 80 KHz or at most 70 KHz. For example, a suitable operating frequency is about 55 KHz.

The displacement amplitude at the distal end of the horn may be in the range 1-200 microns peak-to-peak at the operating frequency.

In the method of operation, the ultrasonic transducer may for example be operated at a power density in the range 10-1000 Wcm⁻². The ultrasonic transducer may for example be operated at a power in the range 1-1000 W.

As mentioned, the openings are formed in one or more of the back mass, front mass and ultrasonic horn arrangement. These are referred to here as the vibrational energy transfer path components of the transducer. Each of said components may have a monolithic construction, i.e. formed from a single piece of material without heterointerfaces (grain boundaries are of course permitted). Accordingly, the requirement that the plurality of openings provide an increased mechanical compliance in a direction along the vibrational energy transfer path for an inventive embodiment arrangement is intended to be compared with a reference arrangement in which the ultrasonic transducer is otherwise identical but in which the openings are replaced with the material of the relevant component. Considering the operating frequency of the transducer of an inventive embodiment arrangement compared with that of a reference arrangement, the operating frequency of the inventive embodiment arrangement may be at least 1 kHz (or at least 2 kHz, or at least 3 kHz, or at least 4 kHz, or at least 5 kHz, or at least 6 kHz, or at least 7 kHz, or at least 8 kHz, or at least 9 kHz, or at least 10 KHz) different (e.g. less) than the operating frequency of the reference arrangement. In turn, this allows inventive embodiment arrangements to have a smaller format than other reference arrangements for the same operating frequency. This means that it is more realistic for such transducers to be located for insertion into the body during surgery, rather than locating the transducers outside the body and using elongate waveguides. In turn, this means that the transducer can be located at the distal side of a flexible joint of a surgical device, allowing more convenient positioning of the transducer.

The length of the transducer (measured from the proximal end of the back mass to the distal end of the horn, along the vibrational energy transfer path) may be not more than 40 mm. The maximum diameter of the transducer (measured in a direction perpendicular to the length) may be not more than 15 mm.

Still further, considering an inventive embodiment arrangement compared with a reference arrangement as described above, the inventive embodiment arrangement may demonstrate one or more figures of merit that are the same as or better than the reference arrangement. For example, Kerr for the inventive embodiment arrangement may be the same as or better than k_{eff} for the reference arrangement. Additionally or alternatively, Qₘ for the inventive embodiment arrangement may be the same as or better than Qₘ for the reference arrangement.

### Development B

Following on from their work leading to Development A, the inventors have carried out further investigations and consider that their innovations in this technical field can additionally be expressed as a further development, here termed Development B. It is intended that any of the following aspects and/or further optional features can be combined, singly or in any combination, with any of the aspects or optional features referred to with respect to Development A.

In a general aspect of Development B, the present invention provides an ultrasonic transducer for surgical applications, the ultrasonic transducer comprising:
a back mass;
a front mass;
an ultrasonic actuator arrangement held between the back mass and the front mass;
an ultrasonic horn arrangement forward of the front mass,
wherein the back mass, ultrasonic actuator arrangement, front mass and ultrasonic horn arrangement are arranged along a longitudinal axis of the transducer,
wherein vibrations generated by the ultrasonic actuator arrangement are conducted into the front mass and into the ultrasonic horn arrangement along a vibrational energy transfer path and are amplitude amplified by the ultrasonic horn arrangement,
and wherein one or more of the back mass, front mass and ultrasonic horn arrangement includes a plurality of openings opening towards the longitudinal axis and intersecting the vibrational energy transfer path and configured to provide an increased mechanical compliance in a direction along the vibrational energy transfer path.

In a first aspect of Development B, the present invention provides an ultrasonic transducer according to the general aspect in conjunction with one or more of the following features.

The transducer may be a Langevin type transducer, such as a bolted Langevin transducer. Such transducers are of interest for surgical applications due to their low frequency and high power operating characteristics.

The ultrasonic actuator arrangement may comprise a piezoelectric material element such as a piezoceramic element. A plurality of such elements may be provided. One or more associated electrodes may be provided in order to conduct a driving signal to the piezoelectric material element(s). Where the front and/or back mass is formed of an electrically conductive material such as metal, the front and/or back mass may provide ground electrical contact(s) for the piezoelectric material element(s).

The vibrational energy transfer path in the back mass, front mass and/or ultrasonic horn may include an annular portion. The annular portion may take the form of a hollow cylinder for example, with the wall of the cylinder circumscribing the longitudinal axis and extending along the vibrational energy transfer path and, in operation, vibrational energy passing along the wall of the cylinder. The plurality of openings intersecting the vibrational energy transfer path may therefore be provided through the wall of the annular portion. The openings are typically through-holes. Although it is possible in principle to use blind holes, the subsequent operation of the transducer is expected not to be as suitable.

The plurality of openings is arranged to increase the mechanical compliance in the direction along the vibrational energy transfer path. Specifically, a longitudinal axial stiffness of the back mass, front mass and/or ultrasonic horn may be modified by the presence of the openings. The axial stiffness affects the resonant frequency at which the longitudinal mode occurs.

Longitudinal-torsional (L-T) mode conversion can occur due to the degeneration of a longitudinal mode into a torsional mode or by the coupling of longitudinal and torsional modes [see, for example, Ultrasonics 52 (2012) 950-988]. In operation, the plurality of openings may provide substantially no longitudinal to torsional mode conversion. For example, the openings may be provided in an achiral array. Additionally, or alternatively, the openings may be provided in a non-helical array. If a helical array is identifiable in the array, then preferably a mirror symmetrical helical array is also identifiable in the array. Avoiding L-T mode conversion preserves the longitudinal mode, which is desirable for soft-tissue cutting.

The achiral nature of the array may be determined by the relative positions of the openings optionally in combination with the shape of each opening. For example, the relative position of each opening and the shape of each opening may be arranged such that the array of openings is superimposable onto a mirror image of itself. The mirror image may be defined with respect to a plane of reflection parallel to the longitudinal axis.

In some embodiments, the plurality of openings is not provided in the ultrasonic horn arrangement. Accordingly, to the extent that the present disclosure proposes that the openings are provided in one or more of the back mass, front mass and ultrasonic horn arrangement, and the transducer optionally including further features disclosed herein, it is expressly to be understood that a modification of this disclosure is that the openings are formed only in the back mass and/or front mass and not in the ultrasonic horn arrangement.

The arrangement of openings may be defined in relation to the geometric centre or centroid of each opening. The geometric centre is the average position of all points along the edge of the opening. The openings (e.g. geometric centres) may be provided in a reflective symmetrical array, such that, for at least part of the array, there may be at least one plane of reflective symmetry parallel to and coincident with the longitudinal axis. Alternatively or additionally, for at least part or parts of the array, there may be at least one plane of reflection symmetry perpendicular to the longitudinal axis.

The openings may be arranged based on a repeating pattern. For example, the openings may be arranged based on a two-dimensional regular lattice mapped onto the surface of the front mass and/or back mass. The regular lattice may comprise a unit cell which is defined by its lattice parameters a, b and B, where a and b are the lengths of the respective edges of the unit cell and β is the angle between the edges. The regular lattice may comprise a unit cell in which β is 90 degrees and/or a is equal to b. Regular lattices such as a square lattice, rectangular lattice, triangular lattice, hexagonal lattice may be suitable.

Alternatively the openings may be arranged substantially randomly, or offset randomly from a notional regular repeating pattern.

There may be three or more openings. For example, the openings may be formed in the front mass and/or the back mass and there may be 4 or more, 6 or more, 8 or more, 10 or more, 12 or more, 16 or more, 20 or more, 24 or more, 30 or more, 40 or more, or 50 or more openings. There are typically fewer than 500 openings, although in some embodiments there may be more openings.

The openings may be longitudinally offset from each other along the longitudinal axis. This longitudinal arrangement may comprise two or more openings. For example, there may be 3 or more, 4 or more, 5 or more, 8 or more, 9 or more, or 10 or more openings which are longitudinally offset from each other.

The openings may be arranged in an axial direction parallel to the longitudinal axis. The openings may be formed in the front mass and/or the back mass and for a plane parallel to and ending at the longitudinal axis coinciding with a maximum total number of openings intersecting the plane, the maximum total number of openings intersecting the plane may be at least 2 openings. For example, there may be at least 3 openings, at least 5 openings, at least 6 openings, or at least 8 openings intersected by the plane. The plane may coincide with the openings through their geometric centres.

Alternatively, or additionally, the openings may be arranged circumferentially. For example, the openings may be formed in the front and/or back mass and for a planar cross section taken perpendicular to the longitudinal axis at a position along the longitudinal axis coinciding with a maximum total number of openings intersecting the plane, the maximum total number of openings intersecting the planar cross-section may be at least 2 openings. For example, there may be 4 or more, 6 or more, 8 or more, 10 or more or 12 or more openings which are intersected by the planar cross-section. The planar cross-section may coincide with the openings through their geometric centres.

Alternatively, or additionally, the openings may be formed in the front and/or back mass and for a planar cross section taken perpendicular to the longitudinal axis at a position along the longitudinal axis coinciding with a maximum total number of openings intersecting the plane, the openings may occupy at least 10% of the circumference of the front mass or back mass, respectively. For example, the openings may occupy at least 20%, at least 30%, at least 40%, at least 50%, or at least 60% of the circumference of the front mass or back mass, respectively.

Openings may be formed in two or more of the back mass, front mass and ultrasonic horn arrangement.

Each opening may have a substantially uniform cross section along its depth. Considering the depth direction of the opening as the direction parallel to the walls of the opening, the depth direction may be substantially perpendicular to the vibrational energy transfer path (e.g. locally in the transducer).

Suitable cross sectional shapes for the openings include circular, oval, elliptical, round, triangular, quadrilateral, rectangular, square, rhombus, pentagonal, hexagonal, pentagonal, octagonal etc. A combination of two or more such shapes may be used for the openings. The openings may have randomly-generated shape. The openings typically have a closed perimeter.

The openings may each have the same size (e.g. the same diameter if the openings have circular cross sectional shape, or other characteristic linear dimension(s) for other shapes). The openings may have the same cross sectional area. Furthermore, the openings may have the same shape as each other, or may be selected from a limited number of shapes (e.g. two, three or four).

The openings may have a cross sectional area of, for example, at least 0.01 mm², at least 0.05 mm², at least 0.1 mm², at least 0.2 mm², at least 0.4 mm², at least 0.6 mm², at least 0.8 mm², at least 1 mm², at least 1.5 mm², or at least 2 mm². There is no particular upper limit on the cross sectional area of the openings, except that a suitable number of openings will need to be fitted to the overall size of the transducer in order to have an effect on the compliance of the relevant component.

The openings may be formed in the front mass and/or the back mass and each opening may comprise a plane of reflection symmetry parallel to the longitudinal axis.

Each opening may comprise a longitudinal length in a direction parallel to the longitudinal axis and a circumferential width in a circumferential direction perpendicular to the longitudinal axis. The circumferential width may be greater than the longitudinal length. For example, the value of the longitudinal length may be a percentage of the circumferential width less than 95%, less than 90%, less than 80%, less than 60%, less than 50% or less than 40%.

An opening may have a vertex angle θ which is bisected by a transverse plane perpendicular to the longitudinal axis. Alternatively, or additionally, an opening may have a vertex angle θ which is bisected by a plane parallel to the longitudinal axis. In some embodiments, the lateral (e.g. circumferential) and longitudinal dimensions of the opening are determined for a given cross-sectional area based on the size of the vertex angle θ.

The openings may be formed by any suitable manufacturing technique, e.g. machining, cutting (e.g. laser cutting) or etching, or by manufacturing the component with net shape or near net shape techniques such as casting, additive manufacture, etc.

The openings may be filled with a filler material. The filler material may have a Young's modulus that is lower (e.g. a factor of at least 5 lower, at least 10 lower, at least 20 lower or at least 50 lower) than the Young's modulus of the material through with the openings are formed. Suitable materials include epoxy materials or other resin-based materials.

The front mass may comprise a proximal portion in contact with the ultrasonic actuator arrangement and a distal portion connected to the ultrasonic horn arrangement and an intermediate portion disposed between the proximal portion and the distal portion. The openings may be provided in the intermediate portion.

The proximal portion and the intermediate portion may have substantially the same outer diameter. For example, the proximal portion and the intermediate portion may be formed integrally with each other. The intermediate portion and the proximal portion may have substantially the same outer diameter. For example, the front mass may comprise a substantially uniform cross-section. For example, the front and/or back mass may be a cylinder having a substantially constant diameter. The uniform cross-section and constant diameter are regarded as such without consideration for variations resulting merely from the presence or formation of openings.

An apparent elastic modulus of the intermediate portion may be not more than 80% of the apparent elastic modulus of a notional reference intermediate portion, the notional reference intermediate portion being identical to the intermediate portion except for the openings not being present.

The length of the transducer (measured from the proximal end of the back mass to the distal end of the horn, along the longitudinal axis) may be not more than 60 mm. For example the length of the transducer may be not more than 40 mm. The maximum diameter of the transducer (measured in a direction perpendicular to the length) may be not more than 15 mm.

The operating frequency of the transducer may for example be at least 1 kHz, at least 5 kHz, at least 10 kHz, at least 20 kHz, or at least 30 kHz or at least 40 KHz. The operating frequency of the transducer may for example be at most 200 kHz, at most 150 kHz, at most 100 kHz, or at most 90 kHz, at most 80 KHz or at most 70 KHz. For example, a suitable operating frequency is about 55 KHz.

The displacement amplitude at the distal end of the horn may be in the range 1-200 microns peak-to-peak at the operating frequency.

In the method of operation, the ultrasonic transducer may for example be operated at a power in the range 10-1000 Wcm⁻².

As mentioned, the openings are formed in one or more of the back mass, front mass and ultrasonic horn arrangement. These are referred to here as the vibrational energy transfer path components of the transducer. Each of said components may have a monolithic construction, i.e. formed from a single piece of material without heterointerfaces (grain boundaries are of course permitted). Accordingly, the requirement that the plurality of openings provide an increased mechanical compliance in a direction along the vibrational energy transfer path for an inventive embodiment arrangement is intended to be compared with a reference arrangement in which the ultrasonic transducer is otherwise identical but in which the openings are replaced with the material of the relevant component. Considering the operating frequency of the transducer of an inventive embodiment arrangement compared with that of a reference arrangement, the operating frequency of the inventive embodiment arrangement may be at least 1 kHz (or at least 2 kHz, or at least 3 kHz, or at least 4 kHz, or at least 5 kHz, or at least 6 kHz, or at least 7 kHz, or at least 8 kHz, or at least 9 kHz, or at least 10 kHz) different (e.g. less) than the operating frequency of the reference arrangement. In turn, this allows inventive embodiment arrangements to have a smaller format than other reference arrangements for the same operating frequency. This means that it is more realistic for such transducers to be located for insertion into the body during surgery, rather than locating the transducers outside the body and using elongate waveguides. In turn, this means that the transducer can be located at the distal side of a flexible joint of a surgical device, allowing more convenient positioning of the transducer.

Still further, considering an inventive embodiment arrangement compared with a reference arrangement as described above, the inventive embodiment arrangement may demonstrate one or more figures of merit that are the same as or better than the reference arrangement. For example, k_{eff} for the inventive embodiment arrangement may be the same as or better than k_{eff} for the reference arrangement.

Additionally or alternatively, Qₘ for the inventive embodiment arrangement may be the same as or better than Qₘ for the reference arrangement.

In a second aspect of Development B, the present invention provides a surgical tool comprising an ultrasonic transducer according to the first aspect of Development B.

In a third aspect of Development B, the present invention provides a method of operation of an ultrasonic transducer according to the first aspect of Development B, the method including applying an electrical signal to the ultrasonic actuator arrangement to generate vibrations to be conducted into the front mass and into the ultrasonic horn arrangement along a vibrational energy transfer path and amplitude amplified by the ultrasonic horn arrangement.

In a fourth aspect of Development B, the present invention provides a method of cutting tissue by ultrasonic cutting using an ultrasonic blade, including a method of operating of an ultrasonic transducer as set out in the third aspect and conducting the vibrational energy to the ultrasonic blade.

The invention includes any combination of the aspects and optional features described except where such a combination is clearly impermissible or expressly avoided.

### SUMMARY OF THE FIGURES

Embodiments and experiments illustrating the principles of the invention will now be discussed with reference to the accompanying figures in which:

FIG. 1 shows the Harmonic® ACER+Shears (Ethicon® Endosurgery, Johnson & Johnson, Cincinnati, Ohio, USA).

FIG. 2 shows a generic Da Vinci® end-effector with EndoWrist® technology.

FIG. 3 shows a generic surgical ultrasonic system with its key components.

FIG. 4 shows a schematic perspective cutaway view of a classic d₃₃-mode Langevin-type ultrasonic transducer.

FIG. 5A is a schematic diagram of a symmetric Langevin transducer.

FIG. 5B is a schematic diagram of an asymmetric Langevin transducer.

FIGS. 6A and 6B show the effect of the piezoceramic stack length and position in terms of efficiency (FIG. 6A), and normalised figure of merit (FIG. 6B) for high power ultrasonics. The figures are from reference [15].

FIG. 7 shows a schematic view of the serpentine electrodes and how they may be arranged in a piezoelectric stack.

FIG. 8 schematically illustrates the longitudinal vibration mode of a rod, with Poisson's effect.

FIG. 9 shows views of different hollow cylinders used to consider the effect of different holes to modify the apparent Young's modulus.

FIG. 10 shows the shape of holes used for the samples shown in FIG. 9 and the characteristic angle θ. Note that the orientation of the holes in this drawing is the same as for FIG. 9 and the longitudinal axis is upright on the page.

FIGS. 11, 12 and 13 show the longitudinal mode frequency as a function of cylinder length for each hole configuration based on different values for angle θ. These graphs also indicate the apparent Young's modulus for the different cylinders modelled.

FIG. 14 shows schematic perspective views of BLT models investigated to assess the effect of hole shape and distribution in the front mass.

FIGS. 15-18 show the effect that changing the aperture angle has on electrical impedance magnitude and phase of the 4 f.p.p. HC-TSM transducers.

FIGS. 19-22 show the effect that changing the aperture angle has on electrical impedance magnitude and phase of the 8 f.p.p. HC-TSM transducers.

FIG. 23 shows a comparison between fitted curves for standard and HC-TSM transducers showing the relationship between L1 fᵣ and front mass (FM) to back mass (BM) length.

FIGS. 24 and 25 respectively show electrical impedance magnitude and electrical impedance phase of standard and HC-TSM 55 kHz tuned transducers.

FIGS. 26-30 show views of an exemplary ultrasonic transducer according to an embodiment of the invention. FIG. 26 shows the transducer in front side perspective view. FIG. 27 shows the transducer in rear side perspective view. FIG. 28 shows the transducer in longitudinal cross sectional perspective view. FIG. 29 shows the transducer in exploded side rear side perspective view. FIG. 30 shows the transducer in front perspective view with the horn displaced.

FIG. 31 shows the velocity amplitude with respect to signal frequency of an exemplary ultrasonic transducer.

FIG. 32 shows the displacement amplitude with respect to signal frequency of an exemplary ultrasonic transducer.

FIG. 33 shows a side perspective view of another exemplary ultrasonic transducer according to an embodiment of the invention.

FIG. 34 shows a side perspective view of another exemplary ultrasonic transducer according to an embodiment of the invention.

FIG. 35 shows the dimensions of five different rhombus shaped holes investigated to assess the effect of varying axial angle θ. Note that in this drawing and subsequent drawings the angle θ is measured at a different vertex compared with the longitudinal axis, in contrast with FIG. 10 for example.

FIGS. 36A-C show schematic perspective views of the front mass structures investigated to assess individual and combined effects of varying the axial angle, the number of holes arranged axially, and the number of holes arranged circumferentially.

FIG. 37 shows the effect that changing the number of holes arranged axially has on the resonant frequency for different arrangements and dimensions of holes.

FIG. 38 shows the effect that changing the number of holes arranged circumferentially has on the resonant frequency for different arrangements and dimensions of holes.

FIG. 39 shows the effect that changing the axial angle θ has on the resonant frequency for different arrangements of holes.

FIG. 40 shows the effect that varying the number of holes arranged axially and circumferentially has on the gain of the first longitudinal mode L1 for each transducer.

FIG. 41 shows the effect that varying the arrangement and dimensions of holes in the front mass has on the resonant frequency compared to the resulting mass of each front mass structure.

FIGS. 42A-B show schematic side perspective views of the standard device model presented as CAD and Wireframe drawings, respectively. FIGS. 42C-D show side perspective views of the standard device model in its contracted and expanded shapes, respectively, of the L1 longitudinal mode.

FIGS. 43A-B show schematic side perspective views of a folded front mass device model presented as CAD and Wireframe drawings, respectively. FIGS. 43C-D show side perspective views of the folded front mass device model in its contracted and expanded shapes, respectively, of the L1 longitudinal mode.

FIGS. 44A-B show schematic side perspective views of a modified front mass device model according to an embodiment of the invention presented as CAD and Wireframe drawings, respectively. FIGS. 44C-D show side perspective views of the modified front mass device model in its contracted and expanded shapes, respectively, of the L1 longitudinal mode.

FIGS. 45A-B show schematic side perspective views of another modified front and back mass device model according to an embodiment of the invention presented as CAD and Wireframe drawings, respectively. FIGS. 45C-D show side perspective views of the modified front and back mass device model in its contracted and expanded shapes, respectively, of the L1 longitudinal mode.

FIGS. 46A-B show schematic side perspective views of a further modified front and back mass device model according to an embodiment of the invention presented as CAD and Wireframe drawings, respectively. FIGS. 46C-D show side perspective views of the second modified front and back mass device model in its contracted and expanded shapes, respectively, of the L1 longitudinal mode.

FIG. 47 shows a front side perspective view of a cylinder showing a suitable random arrangement of openings for incorporation into a front or back mass structure according to another exemplary embodiment of the present invention.

### DETAILED DESCRIPTION OF THE INVENTION

Further background to the present invention, and aspects and embodiments of the present invention will now be discussed with reference to the accompanying figures. Further aspects and embodiments will be apparent to those skilled in the art. All documents mentioned in this text are incorporated herein by reference.

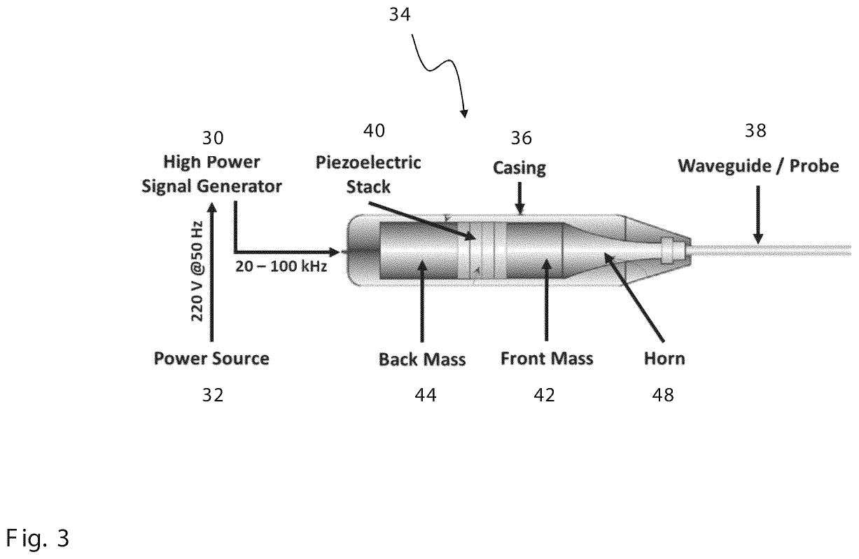

A generic surgical ultrasonic system with its key components is shown in FIG. 3. A high-power signal generator 30 is connected to the power source 32 and controls an ultrasonic transducer, indicated generally by reference number 34. The ultrasonic transducer is a key element of the system, as it converts electrical energy into useful mechanical vibration. A typical operating frequency of the transducer is in the range 20-100 kHz for surgical applications. The ultrasonic transducer is normally located within a casing 36 so it can be handled safely. On one end of the transducer, a probe (waveguide) 38 is attached to guide the wave towards the tissue. As shown schematically in FIG. 3, the transducer includes a piezoelectric stack 40 sandwiched between a front mass 42 and a back mass 44. A horn 46 is located between the front mass 42 and the waveguide 38.

Each of the system's components has its own role and importance, and the design of each part follows certain rules to achieve the desired frequency, performance, and effect in tissue.

The design guidelines for Langevin transducers are the result of many years of successful applications. Numerous books, papers and theses have reported extensively on them. See for example Refs 8, 9 and 10. A brief overview of significant design specifications is given below, as a guide for design considerations.

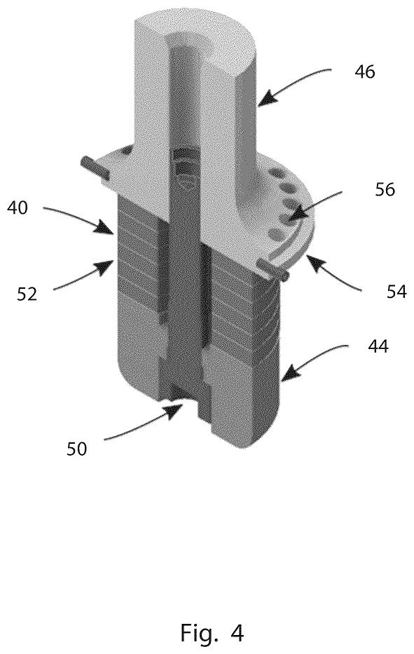

The schematic perspective cutaway view of a classic d₃₃-mode Langevin-type ultrasonic transducer shown in FIG. 4 uses similar reference numbers to FIG. 3 for corresponding features.

The ultrasonic transducer is, in many known devices, inspired by the sandwich configuration introduced by Langevin and Chilowsky, firstly applied in underwater sound projectors in 1918 [1]. The Langevin design, also called ‘Tonpilz’, from the German for ‘singing mushroom’, simply consists of a stack of piezoelectric rings (normally piezoceramic), with intervening electrodes 52, the stack prestressed between a back mass 44 and a front mass 46 with a prestressing bolt 50 [8]. These types of transducers are also known as bolted Langevin transducers (BLTs). In FIG. 4, the front mass includes a flange 54 at the rear end of the front mass and cooling holes 56 formed through the flange 54.

As a starting point for design, the case of a thin rod, where the diameter d is considerably smaller than the length l(d<<l), may be compared to a Langevin transducer to introduce some fundamental concepts. With the assumption of the thin rod, the approximate length of the transducer is half of the wavelength, A, defined in Equation 1,

$$λ = \frac{c}{f} \qquad Equation ( 1 )$$

where c is the speed of sound in the rod and f the desired operational frequency. The speed of sound in a specific material is defined in Equation 2,

$$c = \sqrt{\frac{{E}_{M}}{ρ}} \qquad Equation ( 2 )$$

where E_{M} is the Young's modulus, and ρ the material's density. Equation 3 shows how the natural frequencies, fₙ, for the longitudinal vibrational modes n (n=1,2,3 . . . ) of a thin rod, can be estimated.

$${f}_{n} = \frac{n}{2 l} c \qquad Equation ( 3 )$$

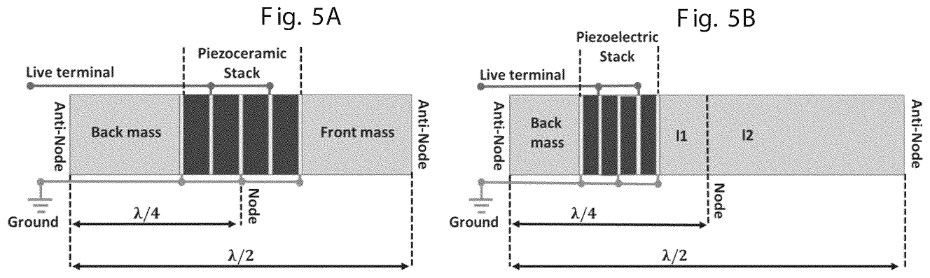

FIG. 5A is a schematic diagram of a symmetric Langevin transducer. If the transducer can be divided centrally at the nodal plane, each half of the transducer, corresponding to λ/4 in length can be studied independently.

FIG. 5B is a schematic diagram of an asymmetric Langevin transducer.

The relationship between the resonant frequency (recalling that ω=2πf), the acoustic impedance, ζ, and the length of both the piezoelectric stack and end masses is expressed by Equation 4.

$$\frac{{ζ}_{m}}{{ζ}_{p}} tan ( \frac{{ω}_{p} {l}_{p}}{{v}_{p}} ) tan ( \frac{{ω}_{m} {l}_{m}}{{v}_{m}} ) = 1 \qquad Equation ( 4 )$$

The subscripts p and m represent piezoelectric and end-mass (either the back or the front mass) respectively. lₚ and lₘ are the thickness of the piezoelectric ring and the length of the end-mass, respectively. ζₘ is the acoustic impedance of an end-mass, which can be obtained from Equation 5:

$${ζ}_{m} = {ρ}_{m} {c}_{m} {A}_{m} \qquad Equation ( 5 )$$

and ζₚ is the acoustic impedance of the piezoelectric element obtained from Equation 6:

$${ζ}_{p} = {ρ}_{p} {c}_{p} {A}_{p} \qquad Equation ( 6 )$$

where A is the cross-sectional area of the component.

Equation 4 can be used to approximate the resonant frequency of a transducer with known dimensions or to find an unknown dimension if the frequency and other parameters are known [12].

The piezoelectric stack 40 is generally made with either d₃₃-mode rings or a d₃₁-mode ring. In high power applications, ‘hard’ piezoceramic such as PZT 4 and PZT 8 is used in even numbers of rings to create the piezoelectric stack, placed between the front and back masses with electrodes in between.

The total length of the piezoelectric stack is chosen to be approximately one quarter of l. However, the choice is influenced by the available driving electronics, as increasing ceramic thickness needs a higher driving electric field and has higher electrical impedance, higher mechanical losses and higher capacitance, resulting in overall higher costs [13].

The centre of the stack is best located at the nodal plane, labelled ‘node’ in FIG. 5A, located in the centre of the transducer in a symmetric design, where the strain is highest. The stack position affects both resonant frequency and vibration amplitude, therefore its location is an important aspect of the design process [14].

Commonly, to allow placement of a supporting flange to attach a casing, the piezoelectric stack is placed away from the nodal position towards the back mass, as shown in FIG. 5B, in an asymmetric configuration. In this configuration, the flange has minimal impact on the vibrational mode. In the asymmetric design, each λ/4 section can be analysed independently and if the half of the transducer including the back mass, the piezoceramic stack, and part of the front mass, l1, is considered, Equation 7 can be used to estimate either the frequency or an unknown dimension of the transducer.

$$\frac{{ζ}_{m}}{{ζ}_{p}} tan ( \frac{{ω}_{p} {l}_{p}}{{v}_{p}} ) tan ( \frac{{ω}_{m} {l}_{m}}{{v}_{m}} ) + \frac{{ζ}_{m}}{{ζ}_{l 1}} tan \text{} ( \frac{{ω}_{l 1} {l}_{1}}{{v}_{l 1}} ) tan ( \frac{{ω}_{m} {l}_{m}}{{v}_{m}} ) + \frac{{ζ}_{p}}{{ζ}_{l 1}} tan ( \frac{{ω}_{l 1} {l}_{1}}{{v}_{l 1}} ) tan ( \frac{{ω}_{p} {l}_{p}}{{v}_{p}} ) = 1 \qquad Equation ( 7 )$$

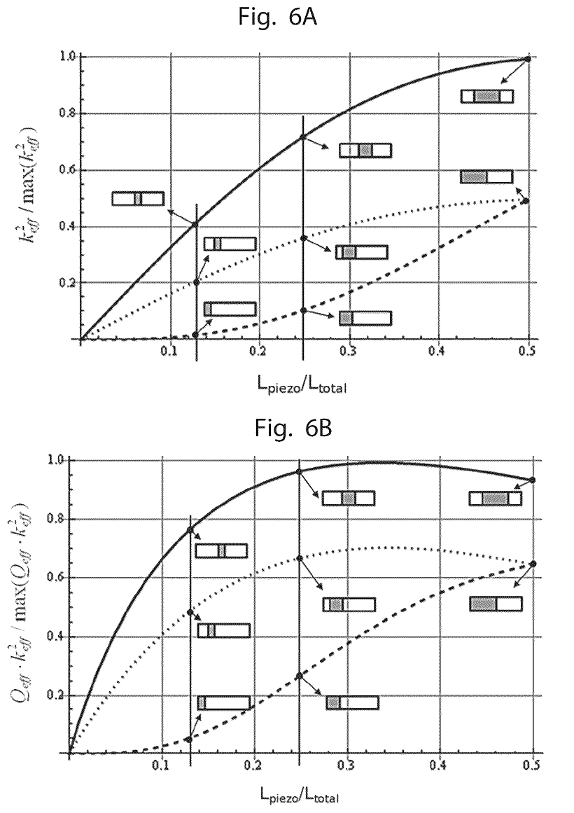

It has been shown by Lierke [15] that the maximum efficiency of the transducer is achieved when the piezoceramic stack is centred and its length is half of the total length of the transducer. The greater the offset from the node, the lower the efficiency, k_{eff}², as shown in FIG. 6A. When considering the figure of merit, normalised by the maximum of k_{eff}²Qₘ, again the best position for the piezoelectric stack is found to be in the centre of the transducer. The normalised figure of merit is above 90% when the ratio L_{piezo}/L_{transducer} is between 0.2 and 0.5, as shown in FIG. 6B [15].

The electrodes are normally chosen with material properties (density, elastic modulus, and acoustic impedance) similar to the piezoelectric materials to avoid unwanted stress concentrations. Note that here we refer to “electrodes” as separate entities from any metallisation formed on the piezoelectric materials.

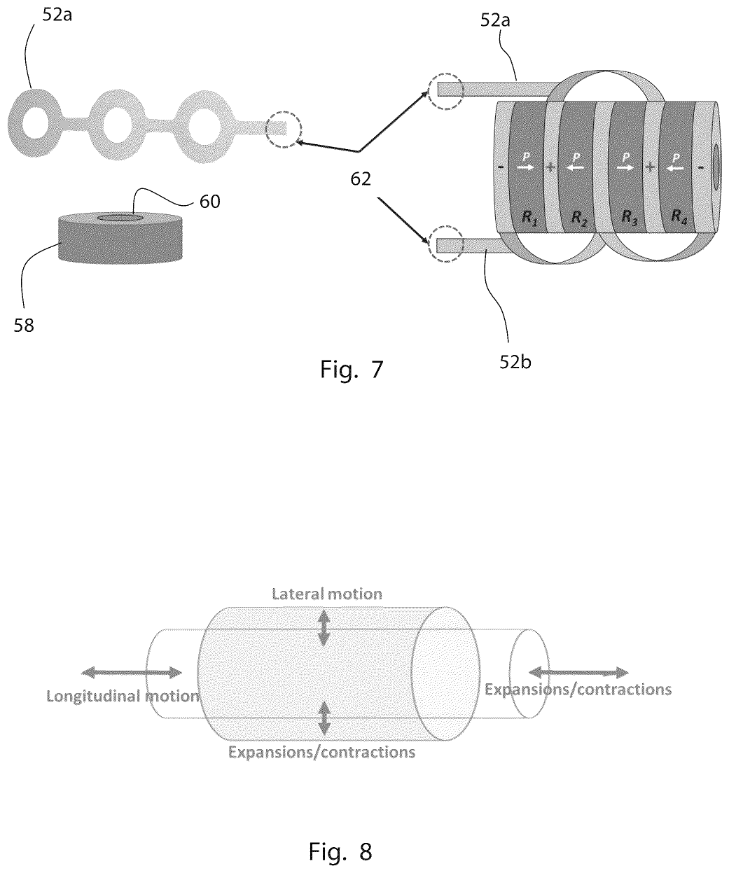

In hand-held transducers, serpentine electrodes 52a, 52b, shown in FIG. 7, may be used in order to provide electrical contact to each side of the respective piezoelectric rings 58. Each piezoelectric ring takes the form of an apertured disk, with the aperture 60 formed through the disk 58 along its principal axis. The left hand part of FIG. 7 shows one electrode 52a and one piezoelectric ring 58 separately. The right hand part of FIG. 7 shows two serpentine electrodes 52a, 52b and four piezoelectric rings R₁, R₂, R₃, R₄ forming the piezoelectric stack. Each serpentine electrode 52a, 52b has electrode ring portions spaced and disposed in order to be located against end faces of respective piezoelectric rings and connecting portions between the electrode ring portions. Such serpentine electrodes are useful in that they can have only limited lateral protrusion and few terminating wires, and no soldering is needed in the vicinity of the rings. The only required soldering is carried out at soldering areas 62, indicated. Cooling fins can also be found in some applications [16].

Metallization on the piezoelectric elements (rings) is normally prepared with 3-10 μm layers obtained through sputtered depositions, electroplating, or special coatings of Cr, Ni, or Au, or other combinations depending on the material suppliers, and these form the interface between the piezoelectric elements and the electrodes.

A prestressing bolt is used to apply and distribute the prestress within the stack. Titanium (or Ti-based alloys) is the preferred material due to its high strength and capability to withstand cyclic loads. The primary reason for prestress is to prevent the piezoceramic from experiencing excessive tensile stress during vibration as piezoceramics are typically almost seven times weaker in tension than in compression. Moreover, the prestress helps to stabilise both the resonance frequency and impedance magnitude and, in addition, it ensures continuity of electrical contact during high level of vibrations [16].

The fundamental desired characteristics of a prestressing bolt are low stiffness, achieved with long bolts, small shank diameter, and low Young's modulus, and high resistance to cyclic loads. The threads of the bolt stretch when the front mass stretches, and the generated friction between the bolt and the front mass can cause undesired heating and subsequent failure (e.g. via fatigue failure) of the thread. The stiffness of a prestressing bolt may be equal to or lower than the front mass stiffness, and may be lower than the piezoceramic stack stiffness. However, long transmission bolts suffer more from mechanical failure [17].

Generally, the required and optimal prestress to apply is not specified and it changes with each design and with the piezoelectric material used. The typical prestresses applied to hard piezoceramics are in the range 25-50 MPa for PZT 4 and 30-79 MPa for PZT 8 [16]. It is important to note that piezoceramic performance degrades with increasing prestress, which causes reductions in piezoelectric coefficients and maximum operating temperature; it may also cause piezoelectric material depolarisation and increase mechanical losses. All these parameters are also known to deteriorate more over time when prestress is applied.

Regarding piezocrystal behaviour under prestress conditions, it has been reported (see references to [20]) that uniaxial pressure, in the range of 0-60 MPa, negatively affects electromechanical properties, with the same consequences as highlighted for piezoceramics. Additionally, piezocrystals may also experience phase transitions under the simultaneous combination of high-power electric field and uniaxial pressure [21].

The back mass has a principal role as an inertial mass, and it also distributes the prestress laterally across the piezoceramic rings. It is normally a solid cylinder, but it has been reported that conic shapes can be used, increasing the bandwidth of the transducer [22]. A rule of thumb for a back mass made of steel indicates that its length should be at least 45% of the diameter of the piezoceramic stack, and its diameter should be at least equal to that of the piezoceramic stack. If other materials are used instead of steel, then the Young's modulus may be used as a comparison, and dimensions adjusted accordingly, in inverse proportion. For example, if a material with half the Young's modulus of steel is used, then the length of the back mass should be doubled (see references [8] to and [23]).

The front mass transfers the vibrational energy to the horn and the probe/blade and often includes a flange which connects to the case. The flange sometimes includes holes for air cooling (see FIG. 4). Sometimes it can be combined with the horn and they can be machined together as it is often made of the same material but, when this is not possible, either spanner holes or wrench flats are provided to connect the front mass and horn with a thread. In surgical tools, wrench flats are preferred as more suitable for small diameter devices. The material used in the front mass is typically more compliant and less dense compared to the material used for the back mass, to facilitate the transfer of the ultrasonic vibration [24].

The horn is a mechanical amplifier which increases the vibration amplitude from the front mass: the vibrational energy travelling through a constant cross-sectional area remains constant; but if the cross-sectional area is progressively reduced along the direction in which the vibrational energy is travelling, then the vibrational energy density and the amplitude increase.

Different horn designs can be used such as stepped, linear or tapered, and exponential, as will be understood by the skilled person. Some horns convert the vibration from the longitudinal mode into a torsional or a transversal mode by using incisions on the surface. In some cases, multiple horns are linked together in cascade, with the amplification sections called ‘boosters’ [25].

The case protects the user from the high voltage and current needed for transducer excitation and also from excess heating. It is designed to clamp the device at its nodal plane so that the fundamental vibrational mode of the device will not be affected.

The waveguide or probe is generally a rod like structure which guides the wave to the tip-end. In devices for soft tissue cutting used in laparoscopic surgery, the tip-end is normally a blade at the end of a long wave guide. It is normally made of Ti6Al4V alloy (90% titanium, 6% aluminium, 4% vanadium, 0.25% iron and 0.2% oxygen) in order to handle the cyclic stresses of the ultrasonic vibrations and at the same time to ensure that it can withstand any loading. The length of the transmission rod is an integer number of half wavelengths, at the operational resonance frequency of the device, and it attaches to the horn at an antinode [26].

The blade at the end of the probe transfers the ultrasonic energy to the tissue and it is shaped according to the desired effect/application. Various blade designs are outlines below.

Suitable blade tips:
(a) for soft-tissue cutting and sealing: THUNDERBEAT® type S, Olympus® Medical [3];
(b) for ultrasonic aspiration: SONOPET® (Stryker®, Kalamazoo, MI, USA);
(c) for precise cutting of bone for reuse elsewhere in the body: SONOPET® Payner® 360 bone tip, (Stryker® [27]) [28];
(d) for bone cutting: SONOPET® bent blade (Stryker®) [29].

As already indicated, an important aspect of the transducer design process is the choice of the materials to be used, which depends on their properties and application requirements.

The Young's modulus, E_{M}, quantifies the stiffness of elastic materials, defined in Equation 8:

$${E}_{M} = \frac{σ}{ϵ} = \frac{\frac{F}{A}}{\frac{d l}{l}} = \frac{F l}{A d l} \qquad Equation ( 8 )$$

as the ratio between stress σ and strain ε, where F is the force applied to the material through a cross sectional area A, and di is the change of length and the initial length is l, along an axis.

The elasticity indicates how a material restores to its original shape after distortion, and the restoring force is proportional to the stress applied. The elasticity is described by Hooke's Law, Equation 9:

$$F = k dl \qquad Equation ( 9 )$$

where k represents the stiffness.

As is widely understood, Hooke's Law is valid only in the elastic region of the stress-strain curve, which is the part of the curve up to the yield strength. A general material may undergo:
tensile stress when normally stretched.
compressive stress when normally compressed.
shearing stress when sheared in plane to the stressed area.

Another important parameter in the choice of materials is the acoustic attenuation. When longitudinal sound waves propagate through a medium, their intensity reduces from the source. Energy loss phenomena are due to scattering and absorption, which are caused by motion of the wave in other directions than the longitudinal and heat generation due to friction. Materials with low acoustic attenuation are preferred; these include light alloys made with metals such as titanium, aluminium and magnesium; heavier material such as brass and tungsten should be avoided if possible. A simple way to look at this is via the longitudinal sound velocity: the faster the speed of sound in a material, the less the energy loss [10].

During cyclic tensile loading, a transducer's components will dynamically deform, experiencing high levels of both stress and strain, dependent on both material properties and the shape of the component, e.g., with sharp corners and step profiles. These forces can concentrate, causing heating and failure e.g., through cracking. A typical design guide indicates that the ultimate tensile strength of each material should be 30% higher than the maximum stress experienced by the tool in operational conditions [30].

Transducer components should also be acoustically matched. The speed of sound in a material is dependent on Eₘ and ρ (Equation 2), but when another material is present, some of the energy will be transmitted forward and some will be reflected back from the interface between the materials, with the possibility that some energy will be converted into a different wave mode.

The amount of energy reflected back is expressed with the reflection coefficient, R_{c}, in Equation 10:

$${R}_{c} = \frac{{ζ}_{2} - {ζ}_{1}}{{ζ}_{2} + {ζ}_{1}} \qquad Equation ( 10 )$$

where ζ₁ and ζ₂ are the acoustic impedance of the first and second media respectively.

The closer the acoustic impedance of the materials, the more energy will be transmitted through the interface, which is desirable for efficient devices. Back and front masses should be made with different materials, with the ‘lower density’ one used for the front mass. This contributes to a larger vibrational amplitude at the front of the transducer. Hence, Equation 11:

$${ζ}_{piezo} = \sqrt{{ζ}_{front mass} {ζ}_{back mass}} \qquad Equation ( 11 )$$

should be respected to achieve maximum energy transmission between the piezoelectric stack and the front mass [10], [31].

Ultrasonic energy can be applied to media to generate different vibrational modes, the simplest of which is the longitudinal mode shown in FIG. 8, where consecutive expansions and contractions along the transducer longitudinal axis are observed with lateral motion simultaneously observed, caused by Poisson's ratio [24].

The longitudinal mode may also be used to generate other modes by modifying the waveguide and/or the horn as noted previously. Asymmetric longitudinal motion can be achieved by using asymmetric blades to add lateral vibrations to the longitudinal motion, making the cutting procedure more effective in certain applications, as previously mentioned for ultrasonic bone-cutting surgical tools.

The cutting mechanisms and dynamics of action depend on the specific surgical task. Cavitation is used in tissue-preserving devices, the direct impact or ‘jackhammer effect’ in bone cutting devices, and the thermal effect is adopted in devices for soft tissue cutting and coagulation.

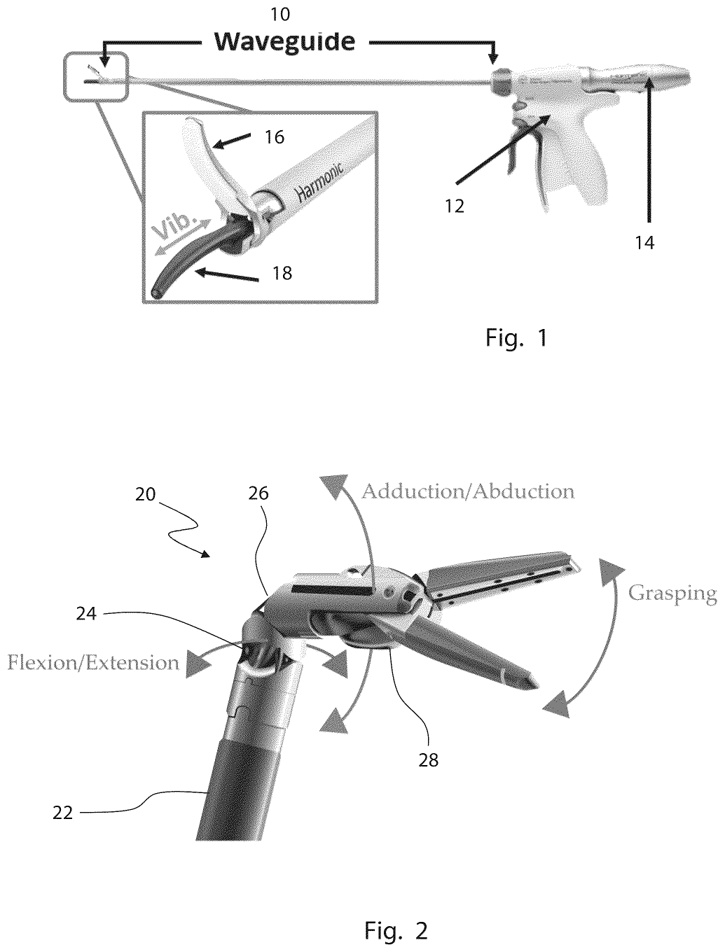

In ultrasonic cutting devices used in soft tissue, the thermal effect is desired, and the tissue is heated to the point of denaturation. The tip of a device to generate this effect has a decoupled bifurcation, as shown in the inset of FIG. 1, allowing the jaw to move and force the tissue onto the ultrasonic vibrating blade. The clamping pressure necessary to close the jaw is applied by the surgeon's hand through a mechanical lever. The induced friction causes the tissue to be heated, denatured and cut in within few seconds, with no bleeding.

The interaction between soft tissue and ultrasonic devices is complex, depending on the protein and water content of the tissue undergoing the surgical procedure. In general, tissue with high water content is easier to cut, whereas tissue with high protein content, such as blood vessels, nerves and connective tissue, requires more energy. The temperature can exceed 100° C. which is sufficient to denature proteins and, if the tissue is heated above its critical necrotic temperature, the damage is irreversible and beyond repair.

The cutting and haemostatic effects are not independent and they happen simultaneously. However, one can be predominant depending on the frequency and vibration amplitude of the blade. Lower frequency and higher vibration result in faster cutting and slower coagulation, while higher frequency and lower vibration cause slower cutting and faster coagulation [25], [26].

The Da Vinci® Surgical System (Intuitive Surgical, Inc.) was introduced along with the EndoWrist® above. The Harmonic® ACE+Shears (Ethicon® Endo-Surgery) is to date the only Da Vinci®-compatible ultrasonic surgical tool. One of the drawbacks of this tool is its lack of maneuverability, i.e. incompatibility with the EndoWrist® technology. This incompatibility is due to several interrelated reasons which are discussed below.

First we consider the operational frequency and device configuration. Ultrasonic surgical devices with haemostatic dissection capability generally operate at about 55 kHz [34]. The operational frequency of a BLT is linked to the device length. Generally, BLTs are designed to operate primarily at their first longitudinal vibrational mode, L1, corresponding to half-wavelength. This puts a constraint on the device length. Additionally, the piezoelectric stack volume and position affect the device's efficiency and functionality. This determines the number of piezoelectric ring elements and their diameter within the stack to achieve the required vibrational amplitude performance. Under these conditions, the device cannot be miniaturised without compromising its operational frequency and without degrading performance, i.e. blade longitudinal vibration amplitude (about 80 μm) [35].

Next we consider the waveguide. From the previous conditions, it emerges that the resulting ultrasonic transducer is effectively too long and large to fit into a laparoscopic port; therefore, a waveguide is used to transfer the vibration from the transducer outside the human body to the end effector (blade) inside the human body. Consequently, this design limits the maneuverability of the blade in view of the need for the waveguide to be long enough to allow the transducer to be outside the body. In order to improve the maneuverability of the blade, it would be useful to include a wristed joint integrated at the end effector of the device. However, this would present an interruption in the waveguide and therefore an interface including a discontinuity. In that case, the ultrasonic wave would not be transmitted towards the blade but would be reflected, resulting in a non-moving blade.

The present inventors have considered whether any previous developments in this field would be useful to assist in the miniaturisation of an ultrasonic transducer that would be useful for surgery.

For example, in [36], BLT optimisation was carried out to miniaturise an ultrasonic scalpel for vessel cutting and sealing and integrate the device with a multi-degree of freedom end-effector for the Micro Hand® S robotic system (Tianjin University, China). A 55 kHz resonating device approximately 50 mm in length, and 10 mm in diameter was developed. The device was successfully integrated and mounted beyond a wrist-like joint and blade displacement of more than 100 μm was reported. Ex vivo experiments were carried out on chicken tissue, demonstrating the functionality and the potential of the device. Effectively, however, the work reported does not present any innovative miniaturisation strategy; since the length of the fabricated device is approximately what is expected for a 55 kHz resonator and is too large for practical laparoscopic surgery.

[37] and [38] report a folded horn transducer, in which the design of the horn allowed a reduction of the horn length by a factor of two whilst maintaining the same operational frequency. While this design reduces the overall length of the device, it does not decouple from the actual horn length.

Flextensional transducers are assembled with a piezoelectric disc sandwiched between two cymbal-shaped metal end caps. Several improvements have been reported for this design, such as the introduction of bolts to prevent failure due to the bonding epoxy layers because of high-power driving. Flextensional transducers can have a relatively small format. However a disadvantage of this design is the possible asymmetry arising from the epoxy bonding layers, which may alter the vibrational mode of the device. Moreover, typical designs exploit the radial mode of a piezoelectric disc, which is not suitable for anisotropic piezocrystals.

A planar ultrasonic silicon scalpel was reported in and [43]. This design used PZT piezoceramics and it was able to achieve a blade vibrational amplitude of about 50 μm at 68 kHz. The device dimensions were length=80 mm, thickness=20 mm and width=22.5 mm, which makes it unsuitable for miniaturisation with the present configuration and material properties of Si. This design was also shown and demonstrated with piezocrystals [23].

Another use of the d₃₁ mode demonstrated the use of piezocrystals for the actuation of a standard needle for anaesthesia [44]. The design incorporated back and front masses and, for this reason, it can be termed a pseudo-Langevin device for its similarity with the piezoelectric ring stack configuration. This design achieves a needle tip displacement of less than 10 μm at 70 VPP. Moreover, the operating frequency of the device is approximately 80 kHz, and the total length without the needle is more than 40 mm. This means that to achieve 55 kHz the device total length must be increased, making this design even less suitable for miniaturisation purposes. A further drawback of these designs is the presence of a bonding layer, in this case made of conductive epoxy, which could fail under high vibrational stresses. Another bonding-related issue with this design can be attributed to the corrupted vibrational mode symmetry which causes stress concentration points which may cause device failure at high vibrational stresses, i.e. detachment from the substrate and cracking.

Next we consider the needs of robotic surgery, relevant to the present disclosure. The superior dissection quality and the speed of sealing of ultrasonic devices, over standard electrocautery, is reported in many studies, e.g. [45]. It is interesting to note that, only a few studies mention the generation of surgical smoke during the procedure [46], [47]. This may reduce laparoscopic visibility and cause delay in the overall procedure time due to endoscope cleaning.

In robotic surgery, ultrasonic dissectors are used predominantly in parenchymal transections to separate the functional tissue of an organ from the connective and supporting tissue, and in lobectomies to remove a lobe or a portion of an organ [5], [6], [48]. Popular robotic procedures, involving the use of ultrasonic dissectors to perform specific intra-operative tasks, include: hepatectomies, splenectomies, enterectomies, adrenalectomies, and thyroidectomies. A study on robot-assisted thyroid surgery compared a wristed bipolar electrocautery instrument (Vessel Sealer Extend, Intuitive® Surgical, Inc.) with an ultrasonic dissector (Harmonic® ACE+Shears, Ethicon® Endo-Surgery).

The direct comparison showed that the use of the ultrasonic device reduced the intra-operative blood loss and improved the dissection margins and speed of seal when compared with the standard electrocautery instrument. However, a higher risk of patient injuries such as burns and involuntary tissue perforations was found with the ultrasonic device, due to the combination of the high temperature of the waveguide and the lack of instrument maneuverability. Therefore, an ultrasonic dissector with a flexible joint, mounted at the end of the robotic shaft beyond the wrist, would address these issues.

The present invention is based on the realisation that it is possible to engineer the stiffness of components of an ultrasonic transducer in order to address some of the design constraints identified above and to decouple fᵣ for the longitudinal mode from the device length. The word “metastructure” is used in the present disclosure (similarly to the concept of a “metamaterial”), indicates a structure engineered to exhibit mechanical properties which differ from those of the bulk material from which the structure is made.

Equation 3 showed the relationship between the natural frequency of the longitudinal mode, the speed of sound and the length of a thin rod. Considering n=1, and replacing c with its mathematical definition (Equation 2), Equation 12 is obtained:

$${f}_{n} = \frac{1}{2 l} \sqrt{\frac{{E}_{M}}{ρ}} \qquad Equation ( 12 )$$

Young's modulus represents the stiffness of a material, therefore rod-like structures made with materials with low E_{M} will vibrate longitudinally at lower frequencies than less compliant (more stiff) materials. Thus, in the context of embodiments of the present invention, it is of interest to change (or tune) the stiffness of a rod-like structure to make it resonate at a desired frequency without altering its length.

A review of mechanical metamaterials shows some strategies to change the mechanical properties of a structure by engineering the unit cells forming the overall lattice. In particular, it is possible to alter the apparent Young's modulus of a structure in one or more directions, typically leading to an increase in anisotropy of the Young's modulus for the structure.

To implement this concept for ultrasonic transducers, a study was performed and results successfully indicated that a transducer cylindrical component can be engineered to modify its apparent Young's modulus and therefore modify the frequency of the longitudinal mode without altering the transducer length. In this study, three different formats for a hollow cylindrical component were considered, each being suitable for use as a front mass of a BLT transducer.

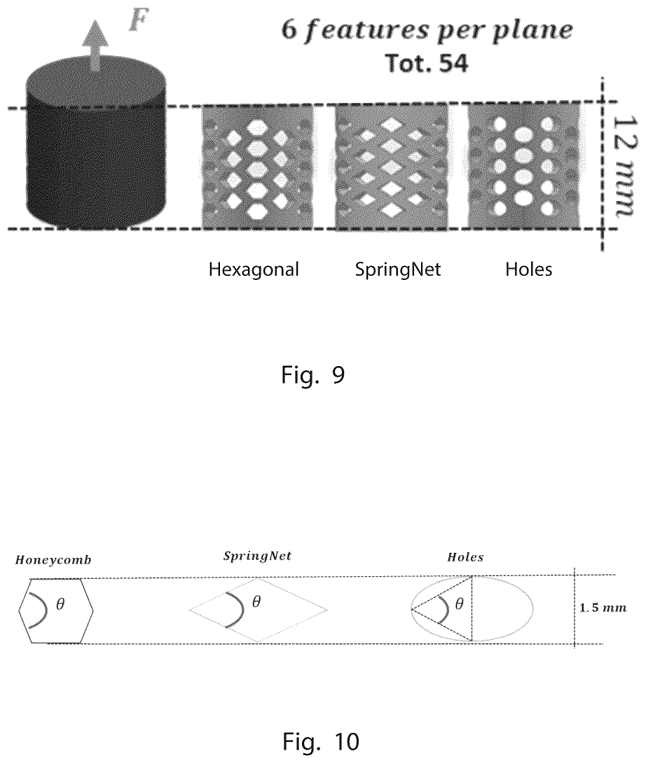

As shown in FIG. 9, the Hexagonal sample used an array of hexagonal through-holes through the wall of the hollow cylinder, with a total number of holes being 54. The arrangement of the holes was based on a hexagonal lattice. Considering the centre of each hole, these centres were arranged so that the centres were each aligned with a plane perpendicular to the principal axis of the cylinder, there being 6 centres per plane, regularly circumferentially spaced around the cylinder. In the drawing, the axial length of the cylinder was 12 mm but this is for illustration only—in the modelling, the axial length of the cylinder was changed in order to show the effect on stiffness of the arrangement of the holes. The force F was applied along the principal axis of the cylinder.

The SpringNet sample shown in FIG. 9 was identical to the Hexagonal sample except for the shape of the holes, which were rhombus shape.

The Holes sample shown in FIG. 9 was identical to the Hexagonal sample except for the shape of the holes, which were oval shape.

The shape of holes for each sample was changed based on FIG. 10. For each hole, angle θ is defined, based on a height of the hole being 1.5 mm measured in a direction parallel to the principal axis of the cylinder. Note that in this part of the disclosure angle θ is defined as shown in FIG. 10. For later parts of this disclosure, angle θ is defined differently.

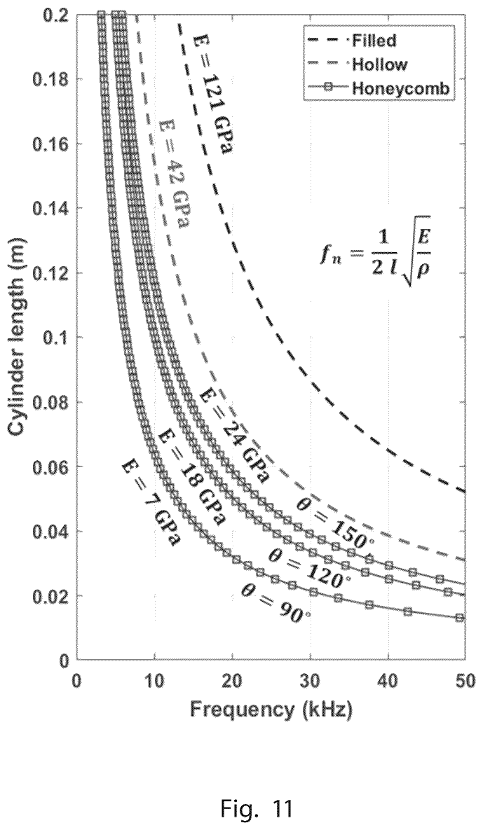

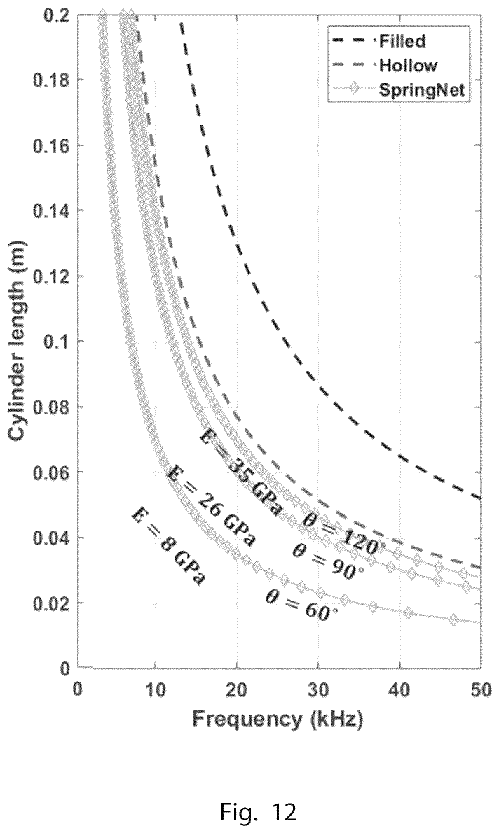

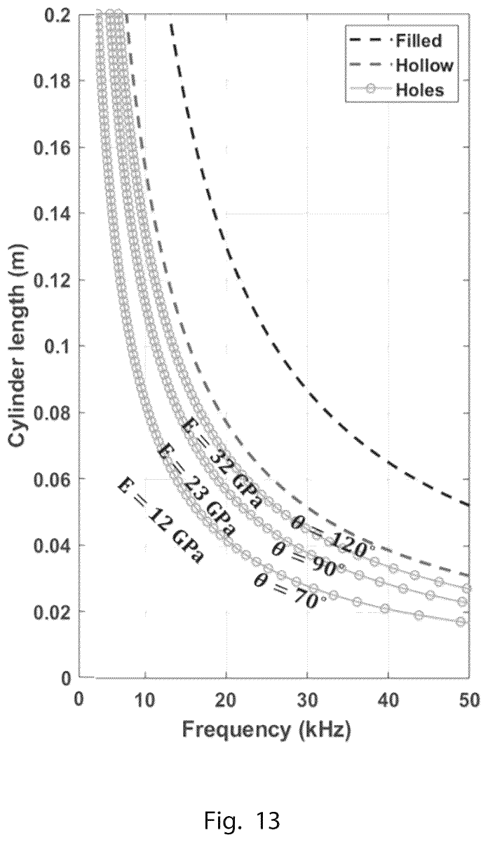

FIGS. 11, 12 and 13 show the longitudinal mode frequency as a function of cylinder length for each hole configuration based on different values for angle θ.

For a non-hollow cylinder, E=121 GPa (i.e. the Young's modulus of the bulk material) and the frequency response is shown by the upper dashed line. For a hollow cylinder, the apparent Young's modulus is 42 GPa and the frequency response is shown by the lower dashed line. Each sample incorporating holes arranged as described has a lower resonant frequency for a particular length of cylinder compared with the hollow cylinder. Or, put another way, adding holes reduces the length of the cylinder needed in order to achieve a particular resonant frequency. Furthermore, decreasing angle θ for each type of hole leads to a reduction in resonant frequency. Note that the annular wall thickness of the hollow cylinder and the samples incorporating holes is the same.

Based on the study reported above, further investigations were carried out based on the honeycomb (HC) shaped holes. This choice was made because the HC showed great capability in lowering the resonant frequency without introducing other vibrational modes in proximity to L1. The abbreviation TSM used herein refers to Tuneable Stiffness Metastructure.

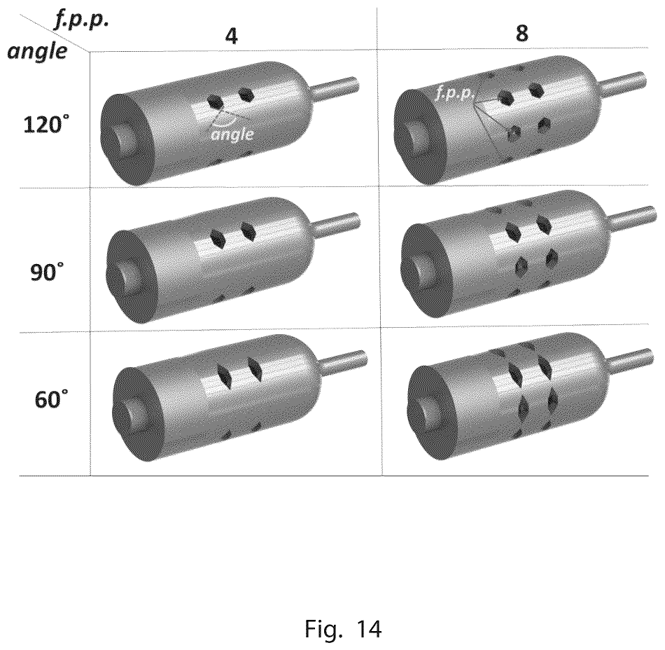

HC-TSM angle θ and the number of HC-TSM features per plane (f.p.p.), were investigated under the hypothesis that these variables should have an impact on the apparent Young's modulus, hence on fᵣ for the L1 mode. All the models investigated in this study are shown in FIG. 14. The results of the design study on HC-TSM are presented and discussed for the L1 mode only, as it represents the vibrational mode of interest for ultrasonic transducers for surgical applications.

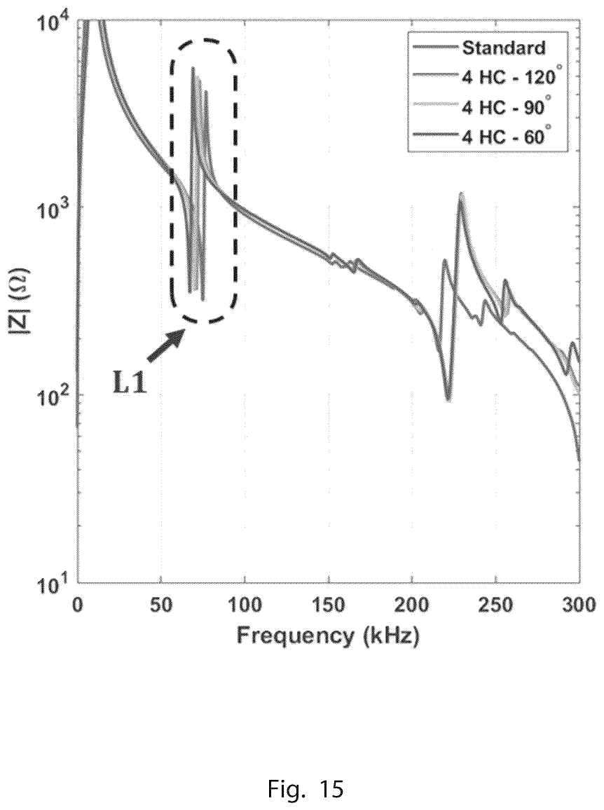

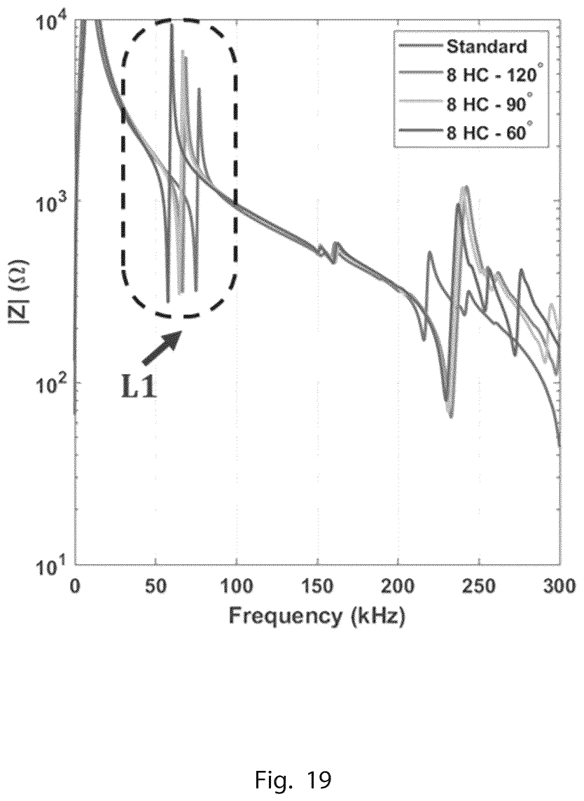

FIGS. 15-18 show the effect that changing the aperture angle has on electrical impedance magnitude and phase of the 4 f.p.p. HC-TSM transducers. FIGS. 19-22 show the effect that changing the aperture angle has on electrical impedance magnitude and phase of the 8 f.p.p. HC-TSM transducers.

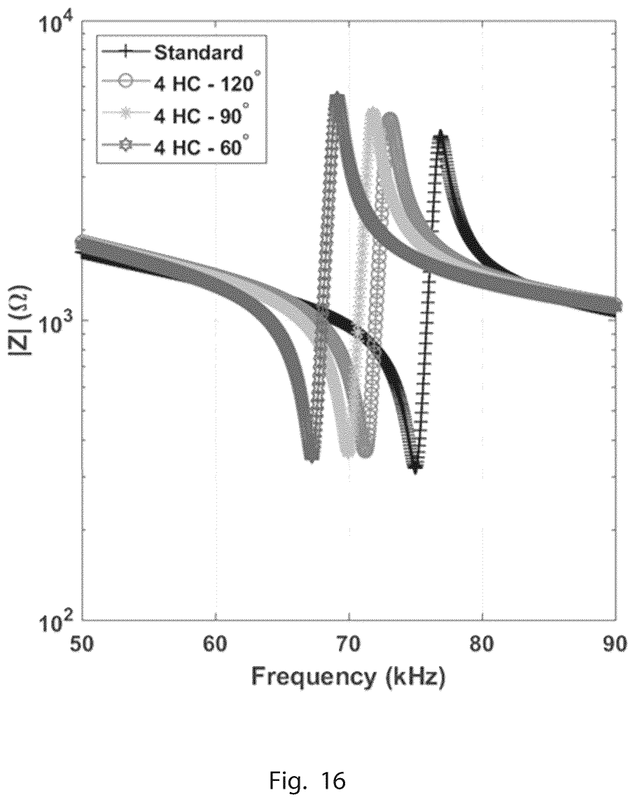

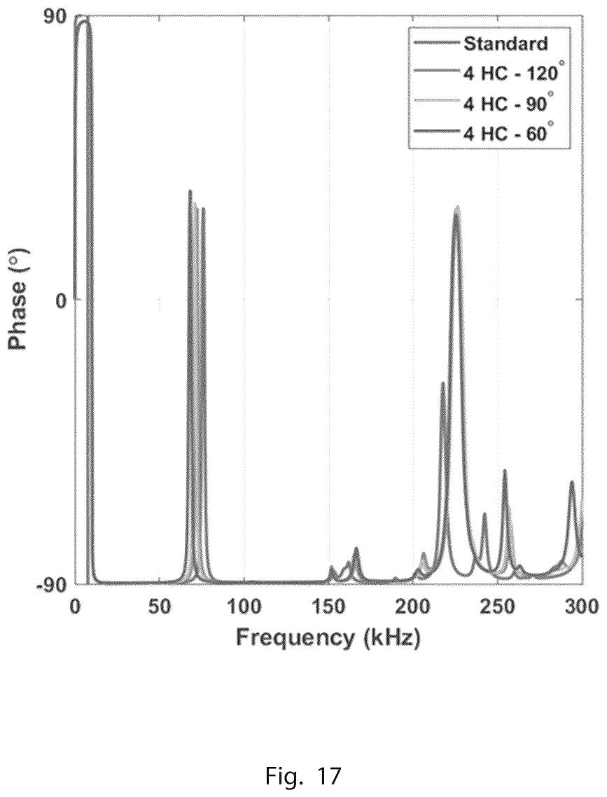

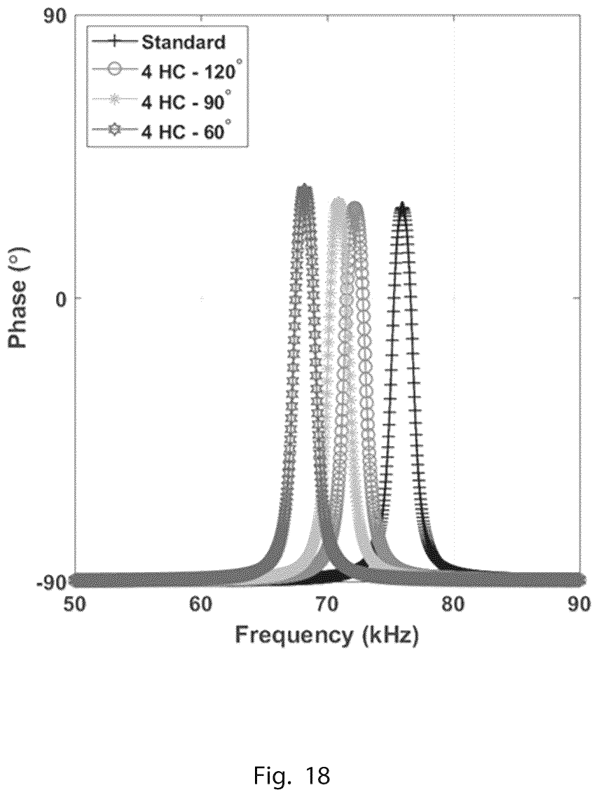

FIGS. 15 and 17 show that in the broader spectrum, 0-300 kHz, changes can be observed in electrical impedance magnitude and phase, and the frequency location of the L1 mode. FIGS. 16 and 18 show the data in the vicinity of the L1 mode. These show that the L1 mode was found to drop in frequency as a consequence of reducing the aperture angle from 120° to 90° and to 60°. The electrical impedance magnitude and phase of the standard transducer are also included for reference.

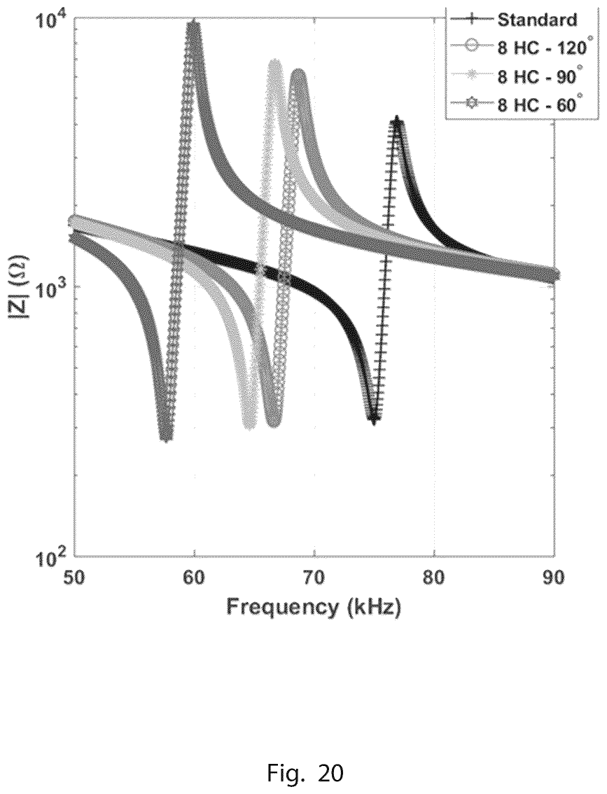

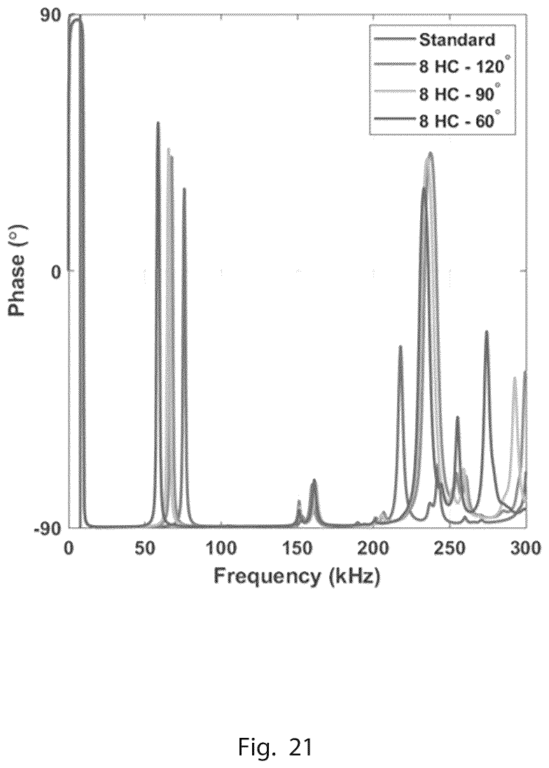

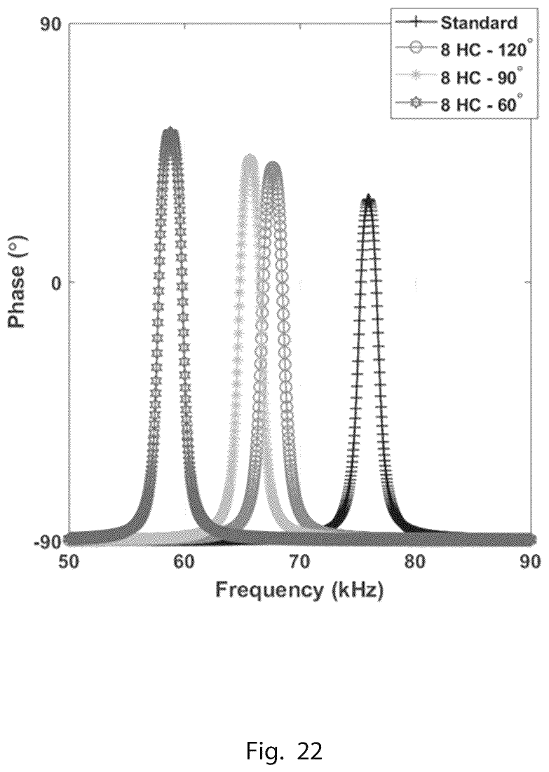

Similarly, FIGS. 19 and 21 show that in the broader spectrum, 0-300 kHz, changes can be observed in electrical impedance magnitude and phase, and the frequency location of the L1 mode. FIGS. 20 and 22 show the date in the vicinity of the L1 mode. These show that the L1 mode was found to drop in frequency as a consequence of reducing the aperture angle from 120° to 90° and to 60°. The electrical impedance magnitude and phase of the standard transducer are also included for reference.

Table 2 shows the effect of the introduction of HC-TSM on the relevant transducer parameters of the L1 mode. The key point that emerges from this design study is that the L1 mode was altered without modifying the length of the transducer. In addition, minor but positive changes were observed in other transducer properties, such as reduced electrical impedance magnitude at fᵣ and improved operational bandwidth.

TABLE 2
Results summary from the L1 mode for the studied
design variables in the HC-TSM transducer.

| FEA | | | fᵣ | Z@ fᵣ | fₐ | Z@ fₐ | k_{eff} | Qₘ |
|---|---|---|---|---|---|---|---|---|
| Model | f.p.p. | angle | kHz | kΩ | kHz | kΩ | / | / |
| Standard | | | 74.99 | 0.32 | 76.92 | 4.11 | 0.22 | 65.88 |
| HC-TSM | 4 | 120° | 71.25 | 0.36 | 73.11 | 4.68 | 0.22 | 65.10 |
| | | 90° | 69.97 | 0.36 | 71.83 | 4.92 | 0.22 | 66.13 |
| | | 60° | 67.27 | 0.35 | 69.17 | 5.49 | 0.23 | 68.29 |
| HC-TSM | 8 | 120° | 66.65 | 0.32 | 68.69 | 6.11 | 0.24 | 70.26 |
| | | 90° | 64.64 | 0.30 | 66.72 | 6.70 | 0.24 | 70.88 |
| | | 60° | 57.67 | 0.28 | 59.90 | 9.28 | 0.27 | 75.28 |

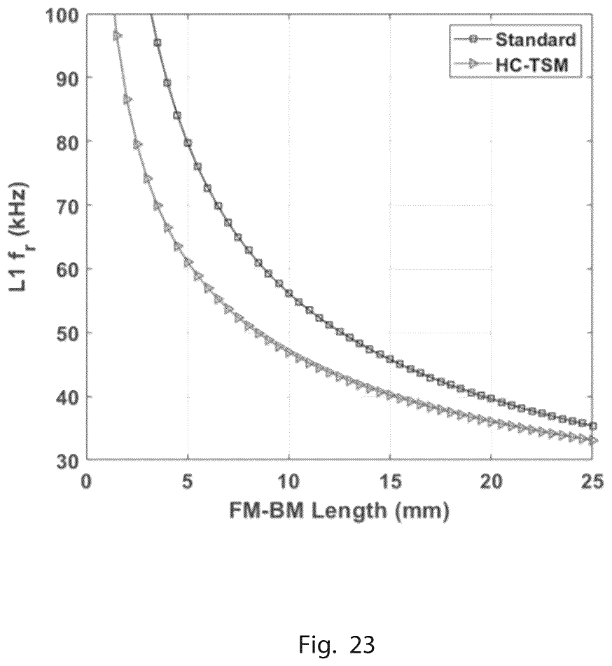

It is therefore possible to make a comparison between a “standard” 55 kHz transducer and an exemplary HC-TSM transducer, where the transducers differ in the construction of the front mass based on the discussion set out above. The most important initial comparison that can be made between the two transducer models is the capability of the HC-TSM device to resonate at the L1 mode with a lower frequency than the standard device despite being of equal length and made from the same materials, as shown in FIG. 23.

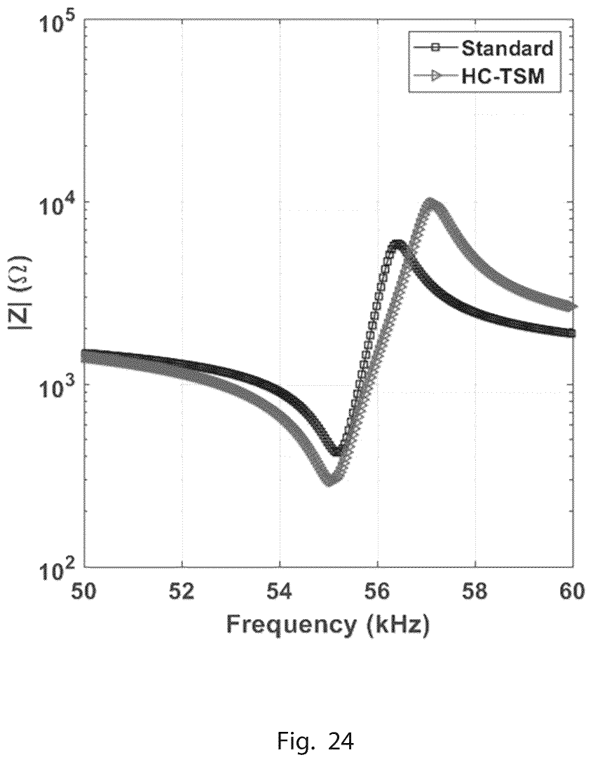

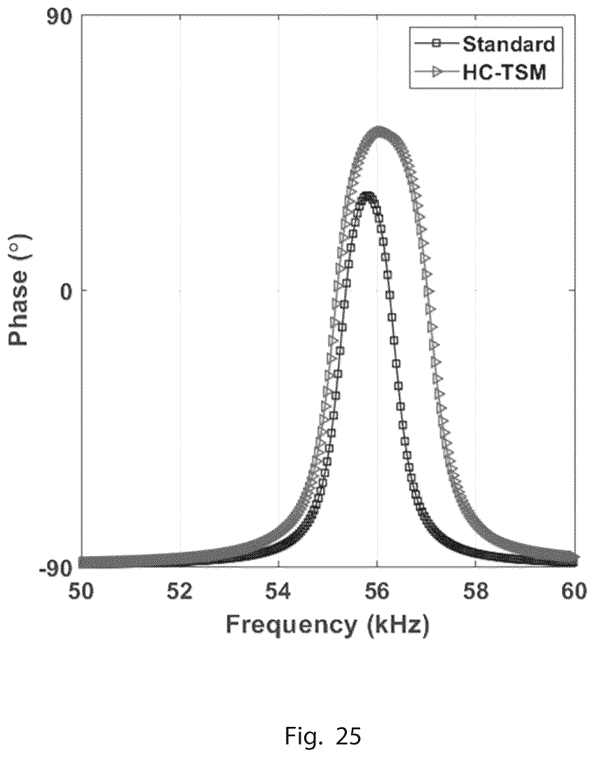

FIGS. 24 and 25 respectively show electrical impedance magnitude and electrical impedance phase of standard and HC-TSM 55 kHz tuned transducers. The electrical impedance magnitude and phase spectra in FIGS. 24 and 25 show that the curve fitting was able to correctly determine the length of FM-BM for a 55 kHz resonating device in both cases.

Table 3 compares the device parameters for the L1 mode of the devices. The introduction of HC-TSM enabled a transducer design which was 20.5% shorter in length than the standard design with approximately the same fᵣ. Moreover, the HC-TSM transducer presents 30% lower electrical impedance at resonance and 28% more bandwidth than the standard design.

TABLE 3
Extrapolated device parameters from simulated electrical impedance
of FIGS. 24 and 25 for 55 kHz-tuned devices for the L1 mode.

| | Tot. | | | | | | |
|---|---|---|---|---|---|---|---|
| | Length | fᵣ | Z@ fᵣ | fₐ | Z@ fₐ | k_{eff} | Qₘ |
| FEA Model | mm | kHz | kΩ | kHz | kΩ | / | / |
| Standard | 44.00 | 55.20 | 421.16 | 56.43 | 5.87 | 0.21 | 78.81 |
| 8 f.p.p, angle | 35.00 | 55.08 | 294.33 | 57.13 | 9.78 | 0.27 | 75.50 |
| 60°, HC-TSM | | | | | | | |

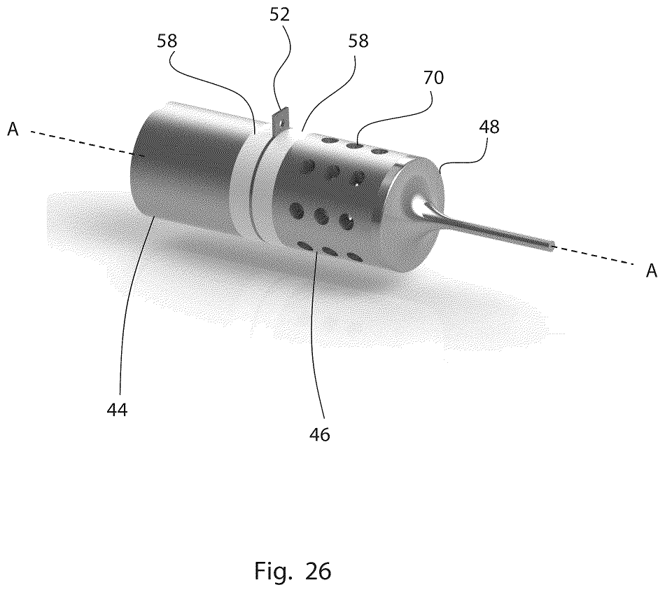

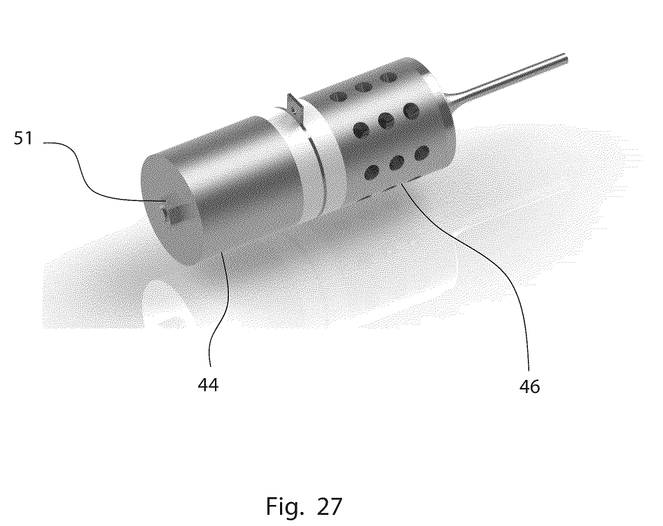

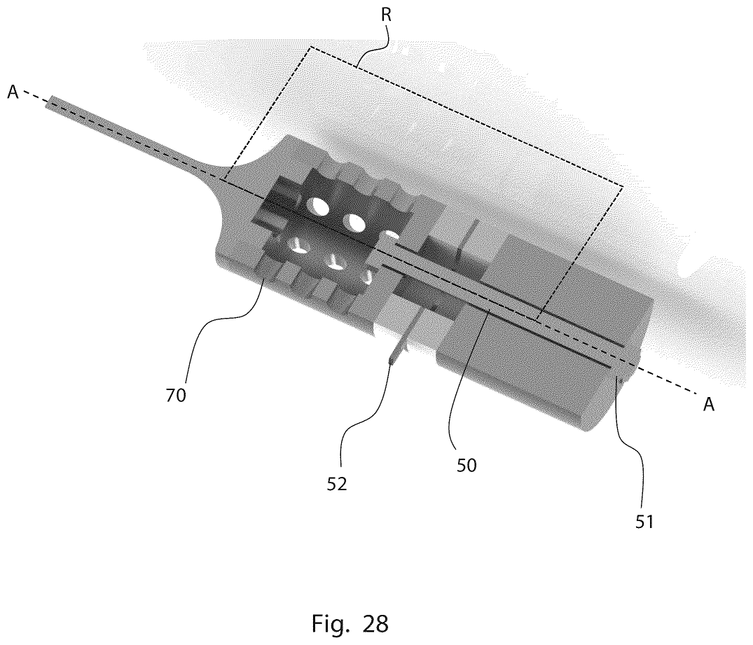

FIGS. 26-30 show views of an exemplary ultrasonic transducer according to an embodiment of the invention. FIG. 26 shows the transducer in front side perspective view. FIG. 27 shows the transducer in rear side perspective view. FIG. 28 shows the transducer in longitudinal cross sectional perspective view.

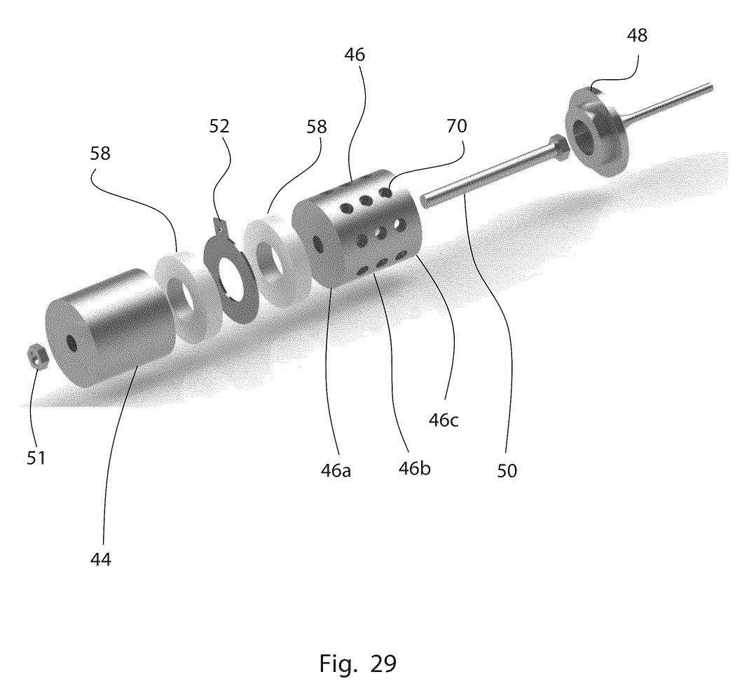

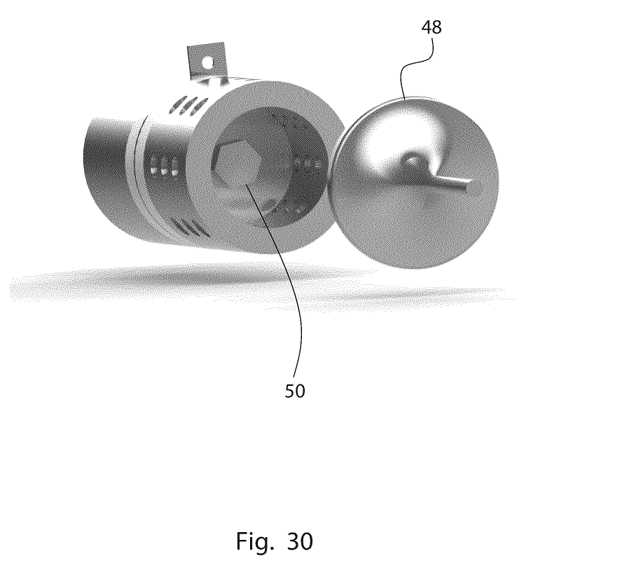

FIG. 29 shows the transducer in exploded side rear side perspective view. FIG. 30 shows the transducer in front perspective view with the horn displaced.

In each of FIGS. 26-30, the transducer has a back mass 44 and a front mass 46 with horn 48 located forwards of the front mass. Two piezoceramic rings 58 of opposing polarity sandwich an electrode 52 to form a piezoelectric stack (ultrasonic actuator arrangement) which is held between the back bass and the front mass by prestressing bolt 50 and nut 51. The front mass has an annular portion which takes the form of an outer cylindrical wall circumscribing the longitudinal axis A, with an arrangement of holes (in this case circular holes) formed through it. In operation, a driving signal is applied to electrode 52 and the front and back masses are earthed, causing oscillation of the piezoelectric rings. The back mass 44, piezoceramic rings 58, electrodes 52, the front mass 46 and horn 48 are arranged along a longitudinal axis of the transducer. This allows vibrations generated by the piezoceramic rings 58 to be conducted into the front mass 46 and into the horn 48 along a vibrational energy transfer path. The vibrations are then amplitude amplified by the horn 48. The circular holes open towards the longitudinal axis and intersect the vibrational energy transfer path. This arrangement provides an increased mechanical compliance in an axial direction parallel to the longitudinal axis and along the vibrational energy path.

Referring in particular to FIG. 29, the front mass 46 includes proximal portion 46a, intermediate portion 46b and distal portion 46c. Proximal portion 46a is in contact with the actuator arrangement. The openings are formed in the intermediate portion 46b. Distal portion 46c is in contact with the horn arrangement. The proximal portion 46a, intermediate portion 46b and distal portion 46c of the front mass 46 are formed integrally with each other and have substantially the same outer diameter, when the effect of the presence of the openings 70 on the outer diameter is ignored.

In operation, the arrangement of openings provides substantially no longitudinal to torsional mode conversion. This preserves the longitudinal mode. This is achieved by the holes being provided in an achiral array, which is superimposable onto a mirror image of itself.

In this embodiment, there are 24 holes in total, which are all formed in the front mass. The holes each have the same depth and cross sectional area. The holes are provided in a reflective symmetrical array (with respect to the geometric centre of each hole), such that there are multiple planes of reflective symmetry parallel to and coincident with the longitudinal axis. One such plane of reflective symmetry is shown in FIG. 28 as the plane along which the cross sectional view has been taken. It will be apparent that there is also a plane of reflective symmetry for a plane perpendicular to the longitudinal axis. The array comprises 3 rows of holes arranged in the axial direction (parallel to the longitudinal axis), wherein each row comprises 8 holes arranged circumferentially. The holes are arranged in a rectangular lattice mapped onto the surface of the front mass. Accordingly, there is a plane of reflection symmetry perpendicular to the longitudinal axis coincident with the second (middle) row of holes.

For a plane parallel to and ending at the longitudinal axis coinciding with a maximum total number of holes intersecting the plane (for example, plane R shown schematically in FIG. 28), the maximum total number of holes intersecting the plane in the front mass in this embodiment is 3. For a planar cross section taken perpendicular to the longitudinal axis at a position along the longitudinal axis coinciding with a maximum total number of holes intersecting the planar cross section, the maximum total number of holes intersecting the planar cross section is 8.

The achievable vibrational amplitudes of standard and HC-TSM 55 kHz transducers were compared. The HC-TSM transducer showed a 33% higher displacement at the blade tip than the standard design for the same driving signal.

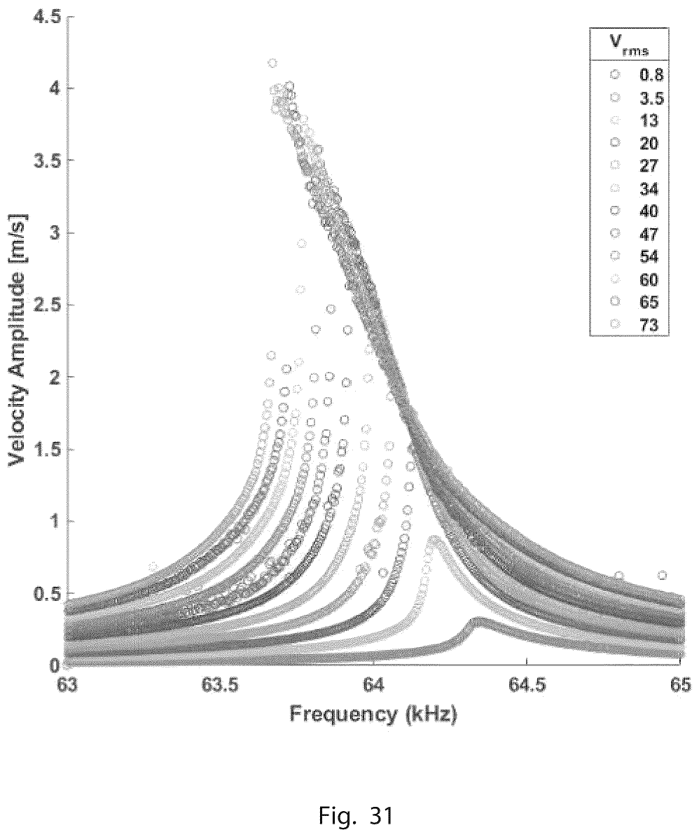

FIG. 31 shows the velocity amplitude with respect to signal frequency of the exemplary ultrasonic transducer. Note that although the legend indicates a signal with Vᵣₘₛ of 0.8 V, there are no data points for this. Instead, the lowest curve is for Vᵣₘₛ of 3.5 V and the curves progressively increase up to Vᵣₘₛ of 73 V.

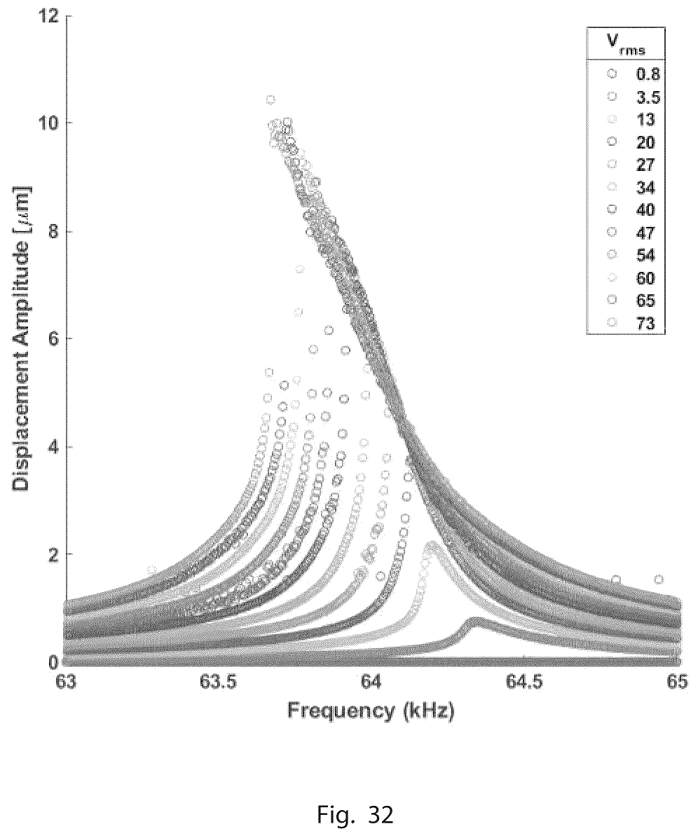

FIG. 32 shows the displacement amplitude with respect to signal frequency of the exemplary ultrasonic transducer. The lowest curve is for Vᵣₘₛ of 0.8 V and the curves progressively increase up to Vᵣₘₛ of 73 V.

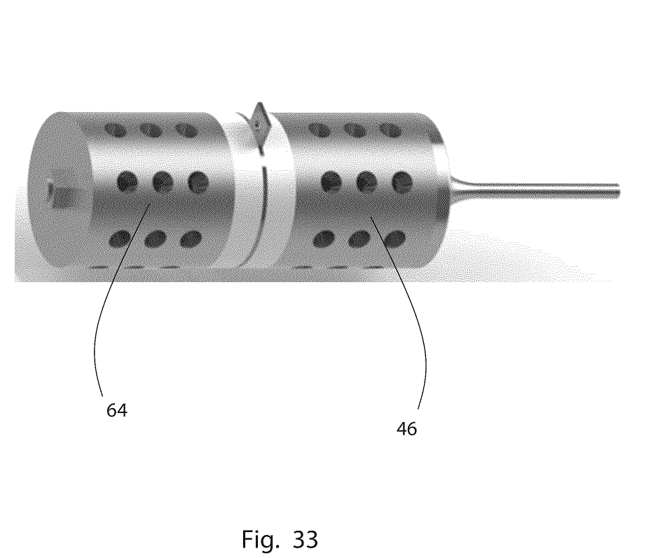

FIG. 33 shows a side perspective view of another exemplary ultrasonic transducer according to a different embodiment of the invention. In this embodiment, the back mass 64 and the front mass 46 each have an outer cylindrical wall with arrangement of circular holes formed therethrough.

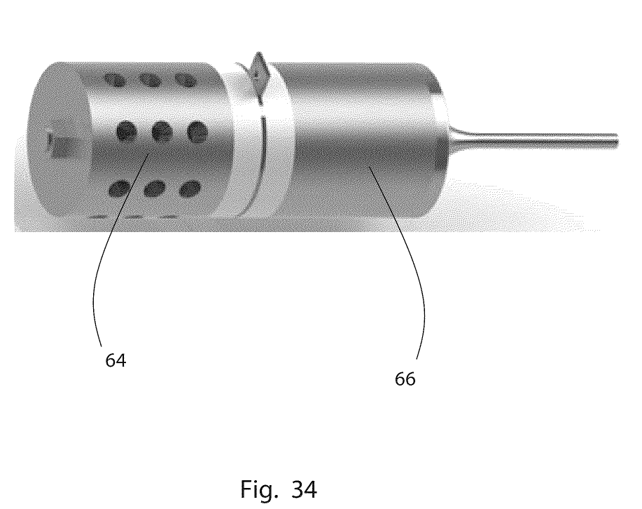

FIG. 34 shows a side perspective of another exemplary ultrasonic transducer according to a different embodiment of the invention. In this embodiment, only the back mass 64 has an outer cylindrical wall with arrangement of circular holes formed therethrough.

In a further study carried out in order to further explore the insights presented above, a number of front masses having differing arrays of openings were proposed to assess the resulting resonant frequencies. The front mass structures were modelled on arrangements of holes formed in a hollow cylindrical front mass, the front mass having an outer diameter of 10.00 mm.

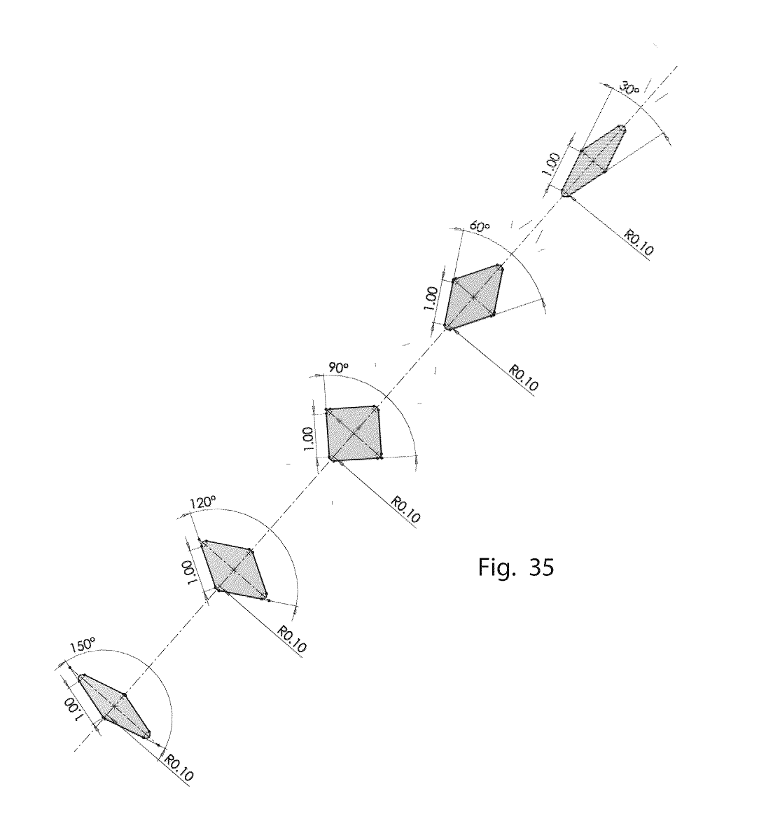

The general shape of each hole used in this further is that of a rhombus (with rounded corners of internal radius of curvature 0.10 mm) with two lines of symmetry. Each hole is aligned to the longitudinal axis of the front mass (and therefore to the longitudinal axis of the transducer) such that one line of symmetry is parallel to the longitudinal axis while the other line of symmetry is perpendicular to the longitudinal axis. In other words, a first pair of opposite interior angles of the rhombus are bisected by a plane parallel to and coincident with the longitudinal axis, while a second pair of opposite interior angles are bisected by a plane perpendicular to the longitudinal axis. Each front mass structure comprises one of five types of rhombus-shaped hole having different dimensions. As illustrated in FIG. 35, each hole type has approximately 1.00 mm side lengths. Accordingly, the hole dimensions are determined by their interior angles and are identified herein by an axial angle corresponding to the first pair of opposite interior angles. The axial angles of the five hole types are 150°, 120°, 90°, 60° and 30°, respectively. Note that the definition of the angles characterising these holes is different to the definition used in FIG. 10. The longitudinal and circumferential dimensions of the rhombus shaped opening are provided in Table 4.

TABLE 4
Longitudinal and circumferential dimensions
of rhombus-shaped hole types.

| | Circumferential | Longitudinal |
|---|---|---|
| Axial | Width, p | Length, q |
| Angle (°) | mm | mm |
| 30 | 0.52 | 1.93 |
| 60 | 1.00 | 1.73 |
| 90 | 1.41 | 1.41 |
| 120 | 1.73 | 1.00 |
| 150 | 1.93 | 0.52 |

The value of the longitudinal length as a percentage of the circumferential width is 57.73% for the holes with a 120° axial angle and 26.79% for the holes with a 150° axial angle, given that each side length of the rhombus-shaped holes is 1.00 mm.

Three parameters were altered between each front mass: the axial angle of the holes, the number of holes in the axial direction (i.e. arranged parallel to the longitudinal axis), and the number of holes in the circumferential direction (i.e. arranged perpendicular to the longitudinal axis).

For simplicity in this further study, the arrangement of the holes was based only on rectangular lattices superimposed onto the cylindrical geometry of the front mass.

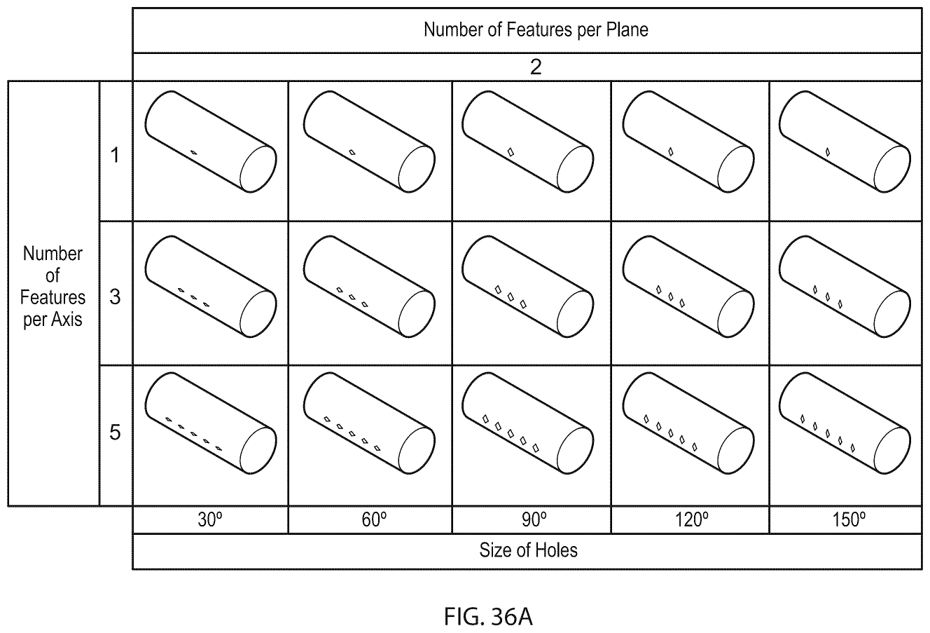

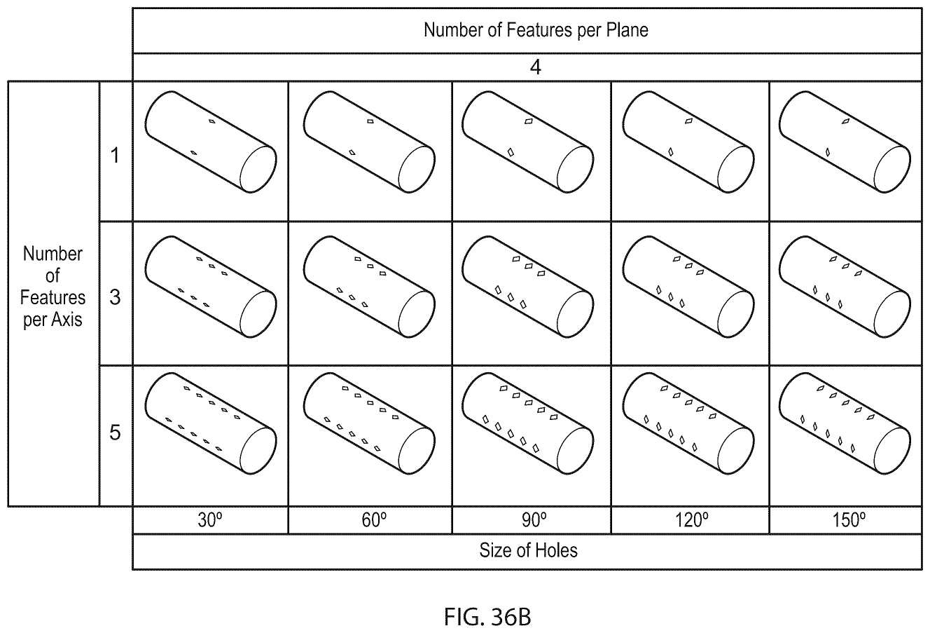

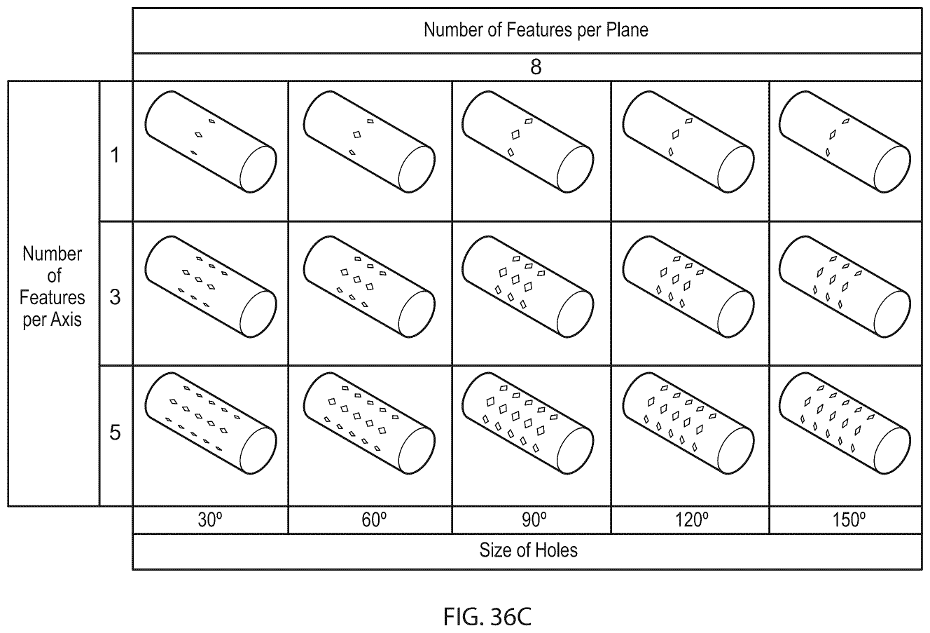

The number of holes in the axial direction was varied between 1, 3 and 5. The number of holes in the circumferential direction was varied between 2, 4 and 8. Therefore, 45 new front mass structures were investigated in total. FIGS. 36A-C show front side perspective views of each front mass structure. FIG. 36A, FIG. 36B and FIG. 36C show sets of the font mass structures having 2 holes, 4 holes and 8 holes arranged circumferentially, respectively.

The following nomenclature was developed for the purposes of identifying each front mass structure: XA_YP_Z, where X, Y and Z represent the number of holes in the axial direction, the number of holes in the circumferential direction, and the axial angle, respectively.

Each front mass has an outer circumference of 31.42 mm. Therefore, for a planar cross section taken perpendicular to the longitudinal axis at a position along the longitudinal axis coinciding with a maximum total number of holes intersecting the plane, the proportion of the circumference occupied by the maximum number of holes (or circumferential fill-factor) for each front mass structure is presented in Table 5 below.

TABLE 5
Maximum proportion of circumference (circumferential
fill-factor) occupied by holes intersecting a planar
cross-section for each front mass structure.

| Front Mass | Circumferential |
|---|---|
| Type | fill-factor (%) |
| _2P_30 | 3.30 |
| _2P_60 | 6.37 |
| _2P_90 | 9.00 |
| _2P_120 | 11.03 |
| _2P_150 | 12.30 |
| _4P_30 | 6.60 |
| _4P_60 | 12.73 |
| _4P_90 | 18.01 |
| _4P_120 | 22.05 |
| _4P_150 | 24.60 |
| _8P_30 | 13.18 |
| _8P_60 | 25.46 |
| _8P_90 | 36.01 |
| _8P_120 | 44.11 |
| _8P_150 | 49.20 |

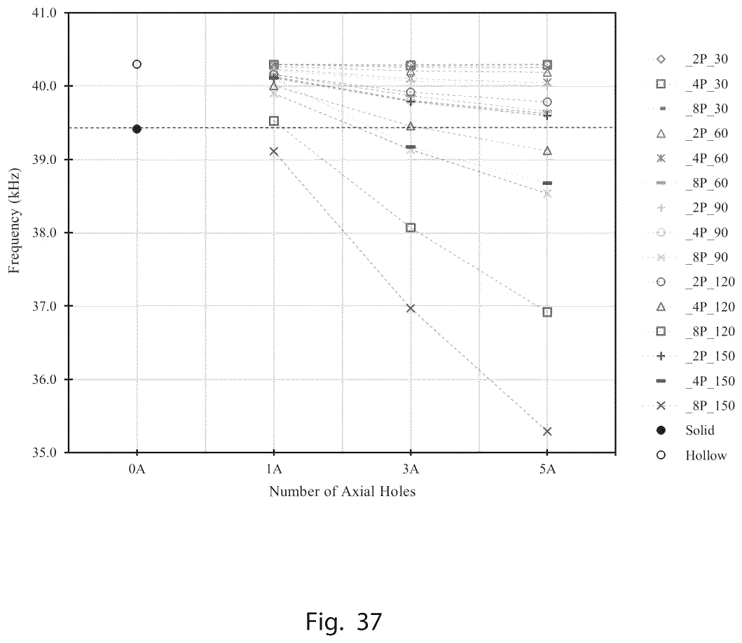

FIG. 37 shows the resonant frequencies of the first longitudinal mode for a varying number of holes in the axial direction. Note that the lines between points are only present for illustrative purposes to demonstrate that the resonant frequency decreases with an increasing number of axially arranged holes. With respect to the data points in the 5A column, the lowest curve corresponds to the _8P_150 structures, followed by _8P_120, _8P_90, _4P_150, _4P_120, _2P_150, _4P_90, _8P_60, _2P_120, _2P_90, _4P_60, _2P_60, _8P_30, _4P_30 and _2P_30, which is the highest curve. The solid data point in the 0A column corresponds to the standard, solid model of a solid front mass without any holes. The horizontal dashed line is present to clarify the divide between an increase and decrease in resonant frequency compared to the standard, solid model. The other data point in the 0A column corresponds to a hollow front mass without holes.

Inspecting the changes in resonant frequency between the 150 1A_8P_150, 3A_8P_150, and 5A_8P_150 devices having the greatest axial angle, an initial 5.5% decrease in resonant frequency is observed, followed by a 4.5% decrease. This trend is exhibited in each series of device with a common angle; thus, increasing the number of axial holes from three to five prompts a smaller decrease in resonant frequency when compared to the change in resonant frequency after increasing the number of holes from one to three, indicating that an inversely proportional relationship exists between the resonant frequency and the number of axially arranged holes.

Hence, further increasing the number of holes in the axial direction will have a diminishing effect on the resonant frequency. The number of axial holes is limited by the dimensions of the transducer, with further increases in the number of holes risking impairment of the structural integrity.

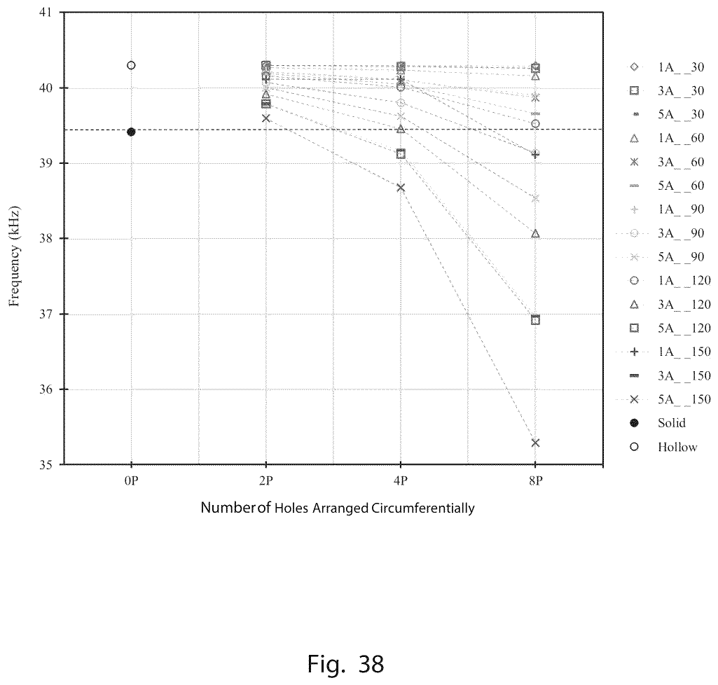

FIG. 38 shows the resonant frequencies of the first longitudinal mode for a varying number of holes in the circumferential direction. As before, note that the data points in the OP column correspond to the standard, solid model and hollow model of a front mass without holes. The horizontal dashed line is present to clarify the divide between an increase and decrease in resonant frequency compared to the standard, solid model. With respect to the data points in the 8P column, the lowest curve corresponds to the 5A__150 structures, followed by 5A__120, 3A__150, 3A__120, 5A__90, 1A__150, 3A__90, 1A_120, 5A_60, 3A__60, 1A__90, 1A__60, 5A__30, 3A__30 and 1A__30, which is the highest curve.

Taking the 5A_X_150 class of devices (where X is either 2P, 4P, or 8P), for example, there is a 2.2% decrease followed by an 8.8% decrease in resonant frequency when increasing the number of holes from two to four and from four to eight, respectively. This trend is exhibited by each of the classes of devices; thus, increasing the number of holes in the circumferential direction from two to four generates a small change in resonant frequency when compared to increasing the number of holes from four to eight. Such a non-linear relationship suggests that further increases to the number of perpendicular holes continue to effectively reduce the resonant frequency. The number of holes, however, is limited by the dimensions of the holes relative to the circumference of the transducer. Similar to the number of holes in the axial direction, increasing the number of holes with impair the structural integrity of the front mass, with stress analyses similarly aiding in understanding the maximum number of holes possible in this direction without adversely affecting the stability of the front mass.

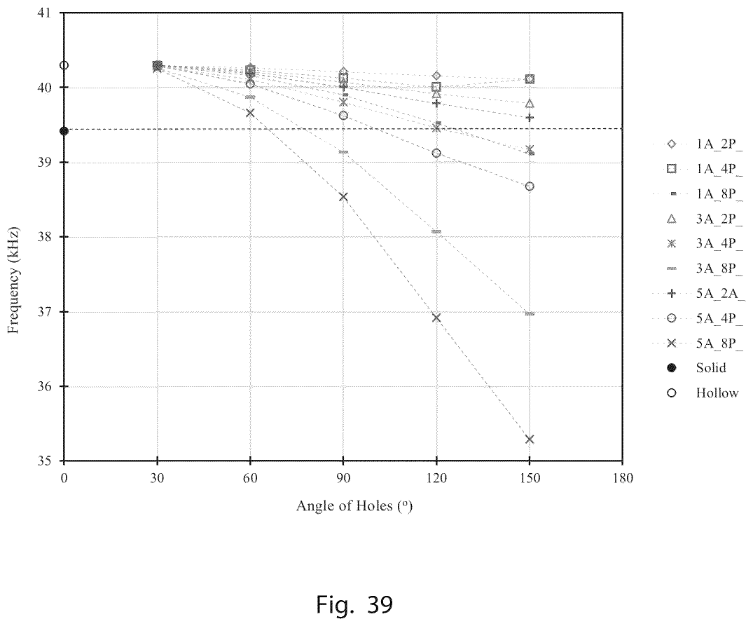

FIG. 39 shows the resonant frequencies of the first longitudinal mode for an increasing axial angle of the holes. As before, note that the data points in the 0° column correspond to the standard, solid model and hollow model of a front mass without holes. The horizontal dashed line is present to clarify the divide between an increase and decrease in resonant frequency compared to the standard, solid model. With respect to the data points in the 150° column, the lowest curve corresponds to the 5A_8P_structures, followed by 3A_8P_, 5A_4P_, 1A_8P_, 3A_4P_, 5A_2P_, 3A_2P_, 1A_4P_and 1A_2P_, which is the highest curve.

The resonant frequency decreases as the angle of the hole increases. Devices that incorporate holes with angles less than 90° exhibit an expected increase in frequency when compared to the standard solid model, as clarified by the dividing, dotted black line. A decrease in resonant frequency was anticipated for devices featuring holes with an axial angle greater than 90°, with eleven of these eighteen devices exhibiting such a decrease. Hence, we may conclude that although increasing the axial angle of the holes is an effective strategy to reduce the resonant frequency of a given device employing an array of holes, it is through the combination of modifying the three parameters related to the array of holes that one would see the greatest decrease in resonant frequency.

The results of this study demonstrate that increasing the number of circumferential holes has the greatest effect on the resonant frequency, followed by the angle of the holes, and the number of axial holes. However, modification to all three of these parameters is advantageous.

The greatest decrease in resonant frequency was observed for the 5A_8P_150 device; a 10.5% decrease in frequency was observed from the solid model resonant frequency to attain a resonant frequency of ˜35 kHz. Comparing this device to a commercially available BLT with a resonant frequency of 40 KHz (manufacturer part number: SMBLTD45F40H) demonstrates that the designed device exhibits a lower frequency despite possessing a similar length (5A_8P_150=53 mm, SMBLTD45F40H=53.75 mm).

To ensure that modification of the front mass structure is a viable strategy to miniaturise ultrasonic transducers, the extent of longitudinal or axial displacement for the modified devices should be similar or greater than that of the standard solid or hollow models. In particular, the transducer should have a suitable gain which is greater than that of the solid and hollow standard models. The gain is determined by the ratio of the maximum axial displacement of each end of the transducer, i.e. the front end (distal end) of the front mass and the back end (proximal end) of the back mass. The axial displacement is the positional change of a part of the ultrasonic transducer in a direction parallel to the longitudinal axis, relative to its equilibrium position. Therefore, the position where the displacement is equal to zero corresponds to the node of the device.

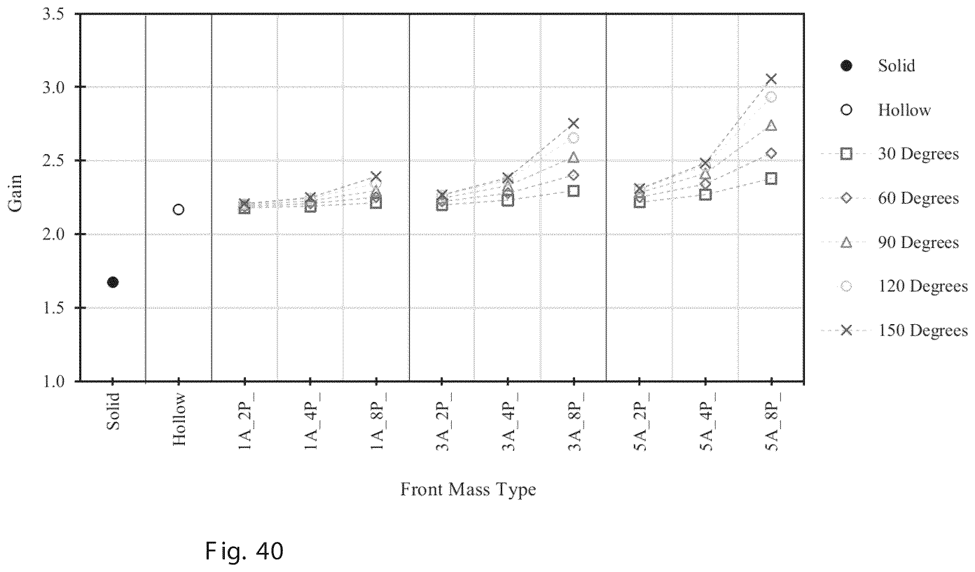

FIG. 40 shows the gains calculated for each transducer device corresponding to the first longitudinal mode L1 of each of the 45 modified front mass structures. The front mass types in the x-axis are divided into groups having a common number of axially arranged holes. It is evident that regardless of the arrangement of holes, each front mass structure results in a gain which is greater than that of the standard, solid model represented by the leftmost data point. Despite this, FIG. 38 suggests that increasing the number of holes arranged axially has a relatively small influence on gain. In contrast, increasing the number of holes arranged circumferentially (i.e. increasing the number of f.p.p.) results in a larger increase in gain—with this effect being observed to a greater extent upon increasing the number of f.p.p. from 4 to 8. The increase in gain that occurs as the axial angle is increased from 30° to 150° is observed substantially more so for devices that utilise a greater number of holes arranged circumferentially.

The greatest increase in gain relative to the standard, solid model occurred for the 5A_8P_150 front mass type, which provided an 82.4% increase in gain. This device also exhibited the greatest decrease in resonant frequency.

It is important to recognise that differences in resonant frequency are caused by changes in mass as well as the influence of the altered front mass structure on mechanical compliance and wave propagation. Therefore, the effect of mass loss on transducer operation was investigated by considering the volume of mass removed for each front mass type for comparison with the resonant frequency of each front mass type.

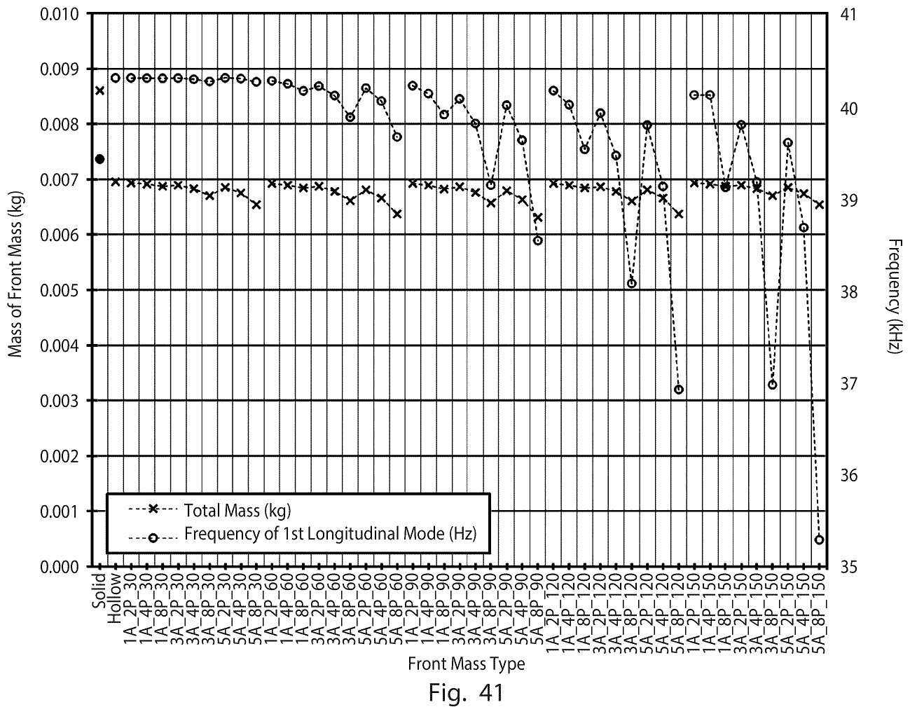

FIG. 41 shows the total mass of the front mass represented by the ‘cross’ data points and the resonant frequency of the first longitudinal mode L1 represented by the ‘circle’ data points, for each front mass type. The front mass types are ordered in the x-axis firstly by axial angle, then by the number of axially arranged holes, followed by the number of circumferentially arranged holes. The groups of points connected by lines represent front mass types which share a common axial angle.

Comparing the masses and resonant frequencies of the 5A_8P_30 (40.2 kHz) and 5A_8P_150 (35.3 kHz), it is observed that, despite possessing the same mass (6.54 g), there is a difference in resonant frequencies of 4.9 kHz, corresponding to a 12.2% decrease in resonant frequency for a 0.0% decrease in mass. It is therefore abundantly evident that the decrease in resonant frequency is driven primarily by changes to the structure of the front mass rather than by variations in mass.

In a further study, three transducer models having different arrangements of openings were proposed for direct comparison with the ‘standard’, solid model and a ‘folded’ front mass model. The standard solid model uses solid front and rear masses except that there is a passage as required through the rear and front mass for the bolt. All five models include a front mass, back mass, two piezoelectric rings, two electrodes and a bolt extended through the back mass and coupled to the front mass. Each transducer has the same total length equal to 67 mm and the same total diameter equal to 15 mm. The material metal making up each device, including the front mass, the back mass and the bolt is Titanium (Ti) and the electrode material for each device is Copper (Cu). Each device model uses the same piezoelectric type PZ26 (MEGGITT) [52].

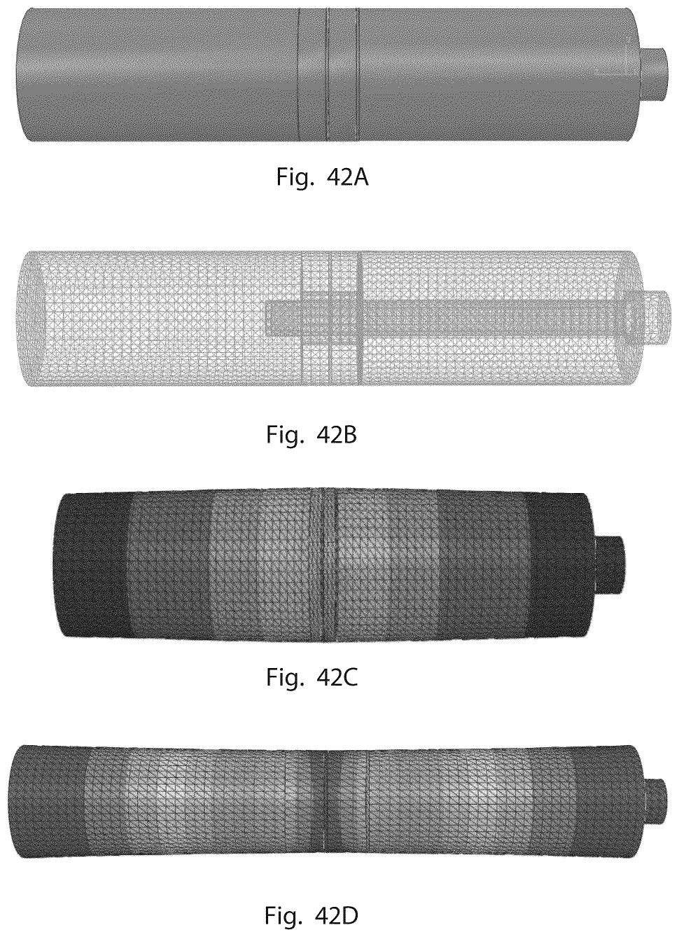

FIGS. 42-46 show schematic illustrations of each transducer device model using separate CAD and Wireframe drawings to present a side perspective view of each model. The contracted and expanded vibration phases at the resonant frequency of the longitudinal mode are shown for each model.

FIGS. 42A-D show the standard model in which the front mass and back are without openings. The contracted shape shown in FIG. 42C corresponds to the ωt=0° phase and the extended shape shown in FIG. 42D corresponds to the ωt=180° phase of oscillation, where w is the resonant frequency of the standard model at the longitudinal mode.

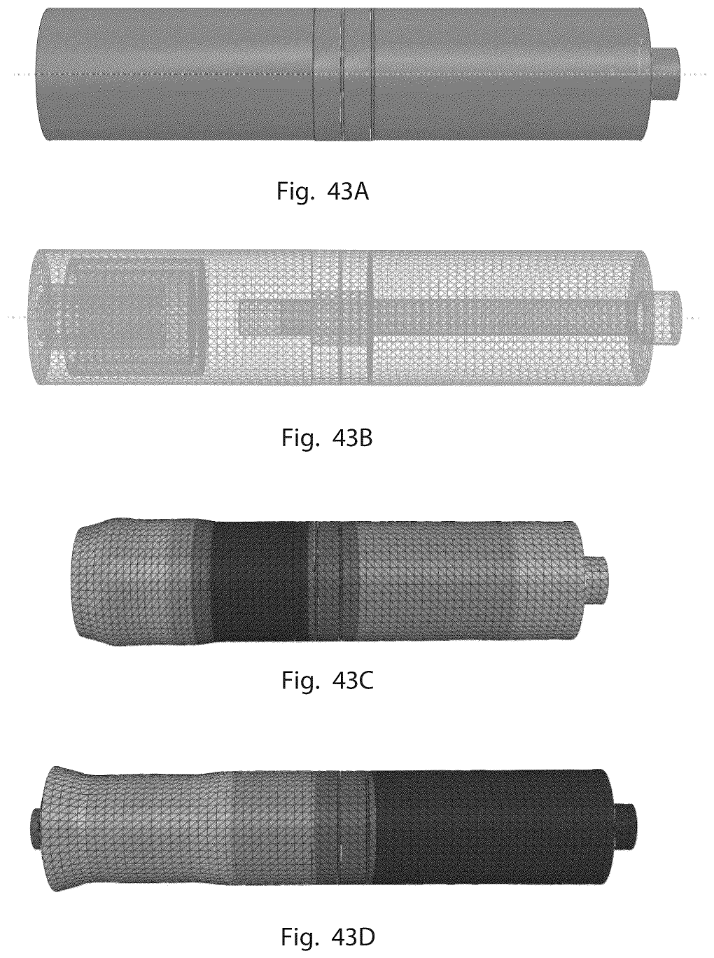

FIGS. 43A-D show the folded front mass model in which the front mass and back mass are without openings, but the front mass comprises a series of concentric inner folds. The series of folds are defined by distinct, overlapping, inner and outer annular cavities. The inner annular cavity projects inwardly from the distal end of the front mass while the outer annular cavity circumscribes the inner annular cavity. An innermost fold comprises a solid cylindrical portion (visible projecting axially in FIG. 43D) which extends axially towards the distal end of the front mass to conduct vibrations forward of the front mass. The contracted shape shown in FIG. 43C corresponds to the ωt=0° phase and the extended shape shown in FIG. 43D corresponds to the ωt=180° phase of oscillation, where w is the resonant frequency of the folded model at the longitudinal mode.

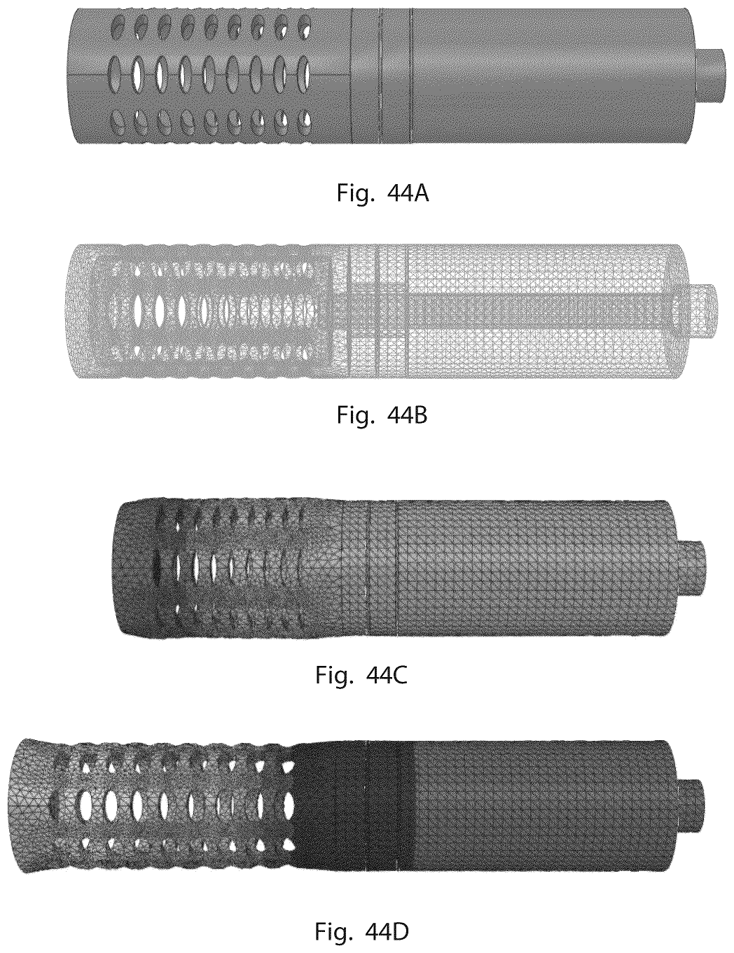

FIGS. 44A-D show a modified front mass model (FM-mod) comprising a solid back mass and an array of oval-shaped holes formed in a hollow front mass. The array comprises 9 rows of holes arranged in the axial direction, wherein each row comprises 8 holes arranged circumferentially. The circumferential dimension of each hole is greater than the corresponding longitudinal dimension of each hole. There are 72 holes in total. The contracted shape shown in FIG. 44C corresponds to the ωt=0° phase and the extended shape shown in FIG. 44D corresponds to the ωt=180° phase of oscillation, where w is the resonant frequency of the FM-mod model at the longitudinal mode.

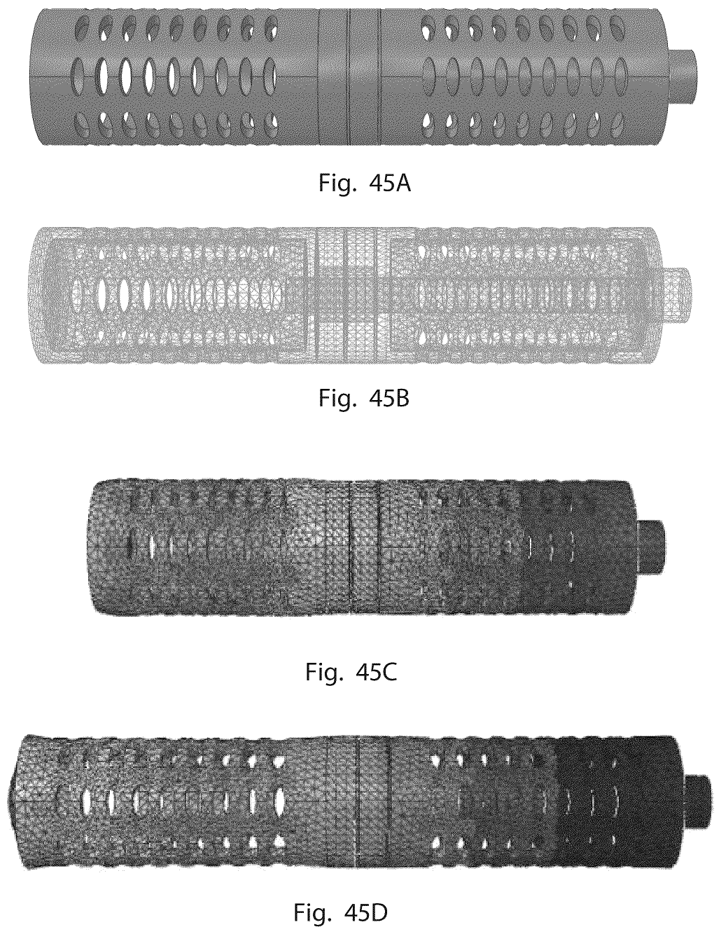

FIGS. 45A-D show a first modified front and back mass model (FM&BM-mod) comprising an array of oval-shaped holes formed in the hollow front mass and a hollow back mass. The circumferential dimension of each hole is greater than the corresponding longitudinal dimension of each hole. There are 144 holes in total, 72 holes in each of the front and the back mass. The array comprises 18 rows of holes arranged in the axial direction, 9 rows in each of the front and back mass, wherein each row comprises 8 holes arranged circumferentially. The holes are arranged in two rectangular lattices mapped onto the surfaces of the front mass and back mass, respectively. Accordingly, for a plane parallel to and ending at the longitudinal axis coinciding with a maximum total number of holes intersecting the plane, the maximum total number of holes intersecting the plane in the front mass is 9. The back mass has the same maximum total number of holes intersecting the plane. For a planar cross section taken perpendicular to the longitudinal axis at a position along the longitudinal axis coinciding with a maximum total number of holes intersecting the planar cross section, the maximum total number of holes intersecting the planar cross section in the front mass and back mass is 8. The contracted shape shown in FIG. 45C corresponds to the ωt=0° phase and the extended shape shown in FIG. 45D corresponds to the ωt=180° phase of oscillation, where w is the resonant frequency of the FM&BM-mod model at the longitudinal mode.

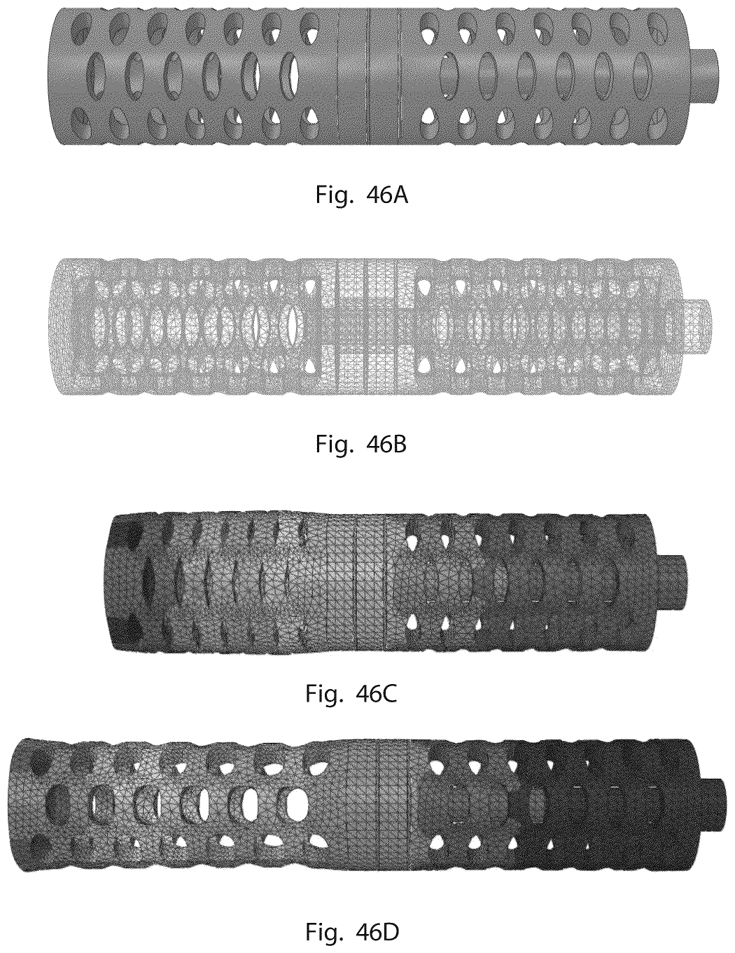

FIGS. 46A-D show a second modified front and back mass model (FM&BM-mod2) comprising an array of oval-shaped holes formed in the hollow front mass and a hollow back mass. The circumferential dimension of each hole is greater than the corresponding longitudinal dimension of each hole. There are 104 holes in total, 52 holes in each of the front and the back mass. The array comprises 26 rows of holes arranged in the axial direction, 13 rows in each of the front and back mass, wherein each row comprises 4 holes. In this model, the holes are arranged in two triangular lattices mapped onto the surfaces of the front mass and back mass, respectively. Accordingly, for a plane parallel to and ending at the longitudinal axis coinciding with a maximum total number of holes intersecting the plane, the maximum total number of holes intersecting the plane in the front mass is 7. The back mass has the same maximum total number of holes intersecting the plane. For a planar cross section taken perpendicular to the longitudinal axis at a position along the longitudinal axis coinciding with a maximum total number of holes intersecting the planar cross section, the maximum total number of holes intersecting the planar cross section in the front mass and back mass is 4. The contracted shape shown in FIG. 46C corresponds to the ωt=0° phase and the extended shape shown in FIG. 46D corresponds to the ωt=180° phase of oscillation, where w is the resonant frequency of the FM&BM-mod2 model at the longitudinal mode.

Table 6 shows the resonant frequency of the L1 mode and the gain from the centre of the piezoelectric stack to the distal end of the front mass for each transducer device model. As shown by the previous design studies, the frequency of the L1 mode was altered without modifying the length of the transducer. Compared to the standard model, all three modified devices provide a lower resonant frequency and a higher gain. The resonant frequency decreased for the folded front mass model relative to the standard model. Notwithstanding this, the FM-mod and FM&BM-mod2 models have lower resonant frequencies than the folded front mass model. This proves that the mechanical compliance of a transducer device may be reduced to a greater extent by having a plurality of openings instead of a folded front mass arrangement.

All three transducer models having arrays of holes formed in the front mass and/or back mass have a larger gain than either the folded front mass model or the standard model. The FM&BM-mod2 transducer device model has the lowest resonant frequency out of the five transducer models presented in Table 6.

TABLE 6
Results for the resonant frequency of the L1
mode and gain in the proposed transducer models.

| | fᵣ | Gain |
|---|---|---|
| Device Model | kHz | / |
| Standard | 32.5 | 5.9 |
| Folded | 24.5 | 13.1 |
| FM-mod | 23.4 | 18.5 |
| FM&BM-mod | 25.1 | 21.2 |
| FM&BM-mod2 | 20.1 | 14.4 |

As an observation, it is noted that the FM&BM-mod device model has a higher resonant frequency than the Folded device model. Without wishing to be bound by theory, it is considered that this is likely to be due to a combination of both higher modal density and the lattice itself dominating the vibrational response. The results are of interest in particular in view of the considerable increase in gain compared to the folded horn structure, given that there is only <1 kHz difference in resonant frequency.

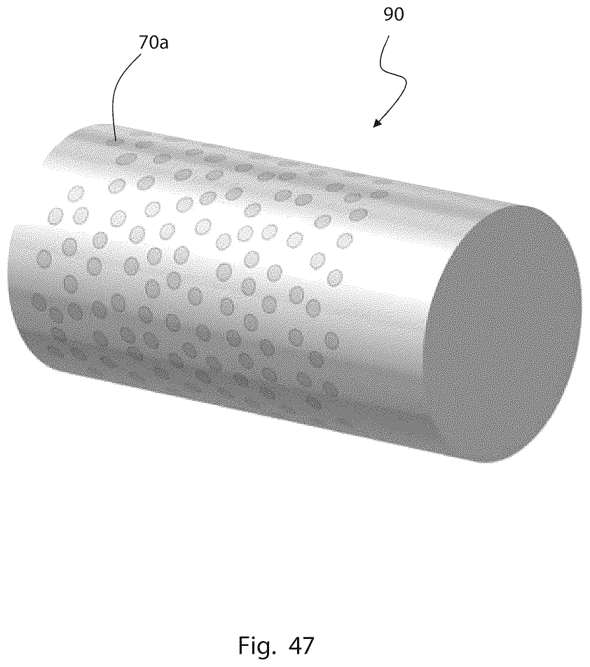

FIG. 47 shows a front side perspective view of a cylinder 90 showing a suitable random arrangement of openings 70a for incorporation into a front or back mass structure according to another exemplary embodiment of the present invention. The holes are arranged substantially randomly but with a minimum distance separating each hole to maintain the structural integrity of the device. Accordingly, the array of holes is intended to provide similar effects to the ordered hole arrangements described above and in particular is intended not to provide substantial longitudinal to torsional mode conversion.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### REFERENCES

A number of publications are cited above in order to more fully describe and disclose the invention and the state of the art to which the invention pertains. Full citations for these references are provided below. The entirety of each of these references is incorporated herein.
[1] D. Zimmerman, “A more creditable way′: The discovery of active sonar, the Langevin-Chilowsky patent dispute and the Royal Commission on Awards to Inventors,” War Hist., vol. 25, no. 1, pp. 48-68, 2018, doi: 10.1177/0968344516651308.
[2] S. K. Longmore, G. Naik, and G. D. Gargiulo, “Laparoscopic Robotic Surgery: Current Perspective and Future Directions,” 2020, doi: 10.3390/robotics9020042.
[3] Olympus, “Thunderbeat,” 2020. https://www.olympus-global.com/technology/technology/thunderbeat/?page=technology_technology (accessed Apr. 28, 2020).
[4] “Ethicon Harmonic Ace.” https://www.ethicon.com/na/epc/code/harh36?lang=en-default.
[5] M. Kroh, Essentials of Robotic Surgery. Springer, 2015.
[6] G. Spinoglio, Robotic Surgery Current Applications and New Trends. Springer, 2015.
[7] S. L. Best and S. Y. Nakada, Minimally Invasive Urology. Springer, 2015.
[8] C. H. Sherman and J. L. Butler, Transducers and Arrays for Underwater Sound. 2013.
[9] D. Stansfield and A. Elliott, Underwater Electroacoustic Transducers: Second Edition. Peninsula Publishing, 2017.
[10] A. Mathieson, “NONLINEAR CHARACTERISATION OF POWER ULTRASONIC DEVICES USED IN BONE SURGERY,” University of Glasgow, 2012.
[11] U. Resonators, “Piezoelectric transducer design,” 2020. http://www.ultrasonic-resonators.org/design/transducers/transducer_design.html #mode_33 (accessed Apr. 26, 2020).
[12] E. A. Neppiras, “The prestressed piezoelectric sandwich transducer,” Ultrason. Int., 1973.
[13] D. A. Deangelis and G. W. Schulze, “Optimizing Piezoelectric Ceramic Thickness in Ultrasonic Transducers,” Proc. 39th Annu. Symp. Ultrason. Ind. Assoc., pp. 1-9, 2010, doi: 10.1109/UIA.2010.5506062.
[14] A. Mathieson, A. Cardoni, N. Cerisola, and M. Lucas, “The Influence of Piezoceramic Stack Location on Nonlinear Behavior of Langevin Transducers,” vol. 60, no. 6, pp. 1126-1133, 2013.
[15] E. G. Lierke, W. Littmann, T. Morita, and T. Hemsel, “Various Aspects of the Placement of a Piezoelectric Material in Composite Actuators, Motors, and Transducers,” vol. 57, no. 4, pp. 933-937, 2010, doi: 10.3938/jkps.57.933.
[16] D. A. Deangelis and G. W. Schulze, “Optimizing Piezoelectric Crystal Preload in Ultrasonic Transducers,” 2009 38th Annu. Symp. Ultrason. Ind. Assoc., pp. 1-6, doi: 10.1109/UIA.2009.5404031.
[17] D. A. Deangelis, G. W. Schulze, and K. S. Wong, “Optimizing piezoelectric stack preload bolts in ultrasonic transducers,” Phys. Procedia, vol. 63, pp. 11-20, 2015, doi: 10.1016/j.phpro.2015.03.003.
[18] X. Liao et al., “Functional piezocrystal characterisation under varying conditions,” Materials (Basel)., vol. 8, no. 12, pp. 8304-8326, 2015, doi: 10.3390/ma8125456.
[19] D. Viehland, “Effect of Uniaxial Stress Upon the Electromechanical Properties of Various Piezoelectric Ceramics and Single Crystals,” J. Am. Ceram. Soc., vol. 89, no. 3, pp. 775-785, 2006, doi: 10.1111/j.1551-2916.2005.00879.x.
[20] Z. Qiu et al., “Characterization of piezocrystals for practical configurations with temperature-and pressure-dependent electrical impedance spectroscopy,” IEEE Trans. Ultrason. Ferroelectr. Freq. Control, vol. 58, no. 9, pp. 1793-803, 2011, doi: 10.1109/TUFFC.2011.2016.
[21] P. Finkel, M. Staruch, A. Amin, M. Ahart, and S. E. Lofland, “Simultaneous Stress and Field Control of Sustainable Switching of Ferroelectric Phases,” Sci. Rep., vol. 5, p. 13770, 2015, doi: 10.1038/srep13770.
[22] B. W. L. Puskas and T. Piazza, “Designer Waveforms: Ultrasonic Technologies to Improve Cleaning and Eliminate Damage,” no. August, pp. 1-9, 2001.
[23] M. R. Sadiq, “Enhanced Biopsy and Regional Anaesthesia through Ultrasound Actuation of a Standard Needle By Muhammad Rohaan Sadiq,” no. February 2013.
[24] K. F. Graff, Power ultrasonic transducers: Principles and design. Elsevier Ltd., 2015.
[25] M. Lucas and A. Mathieson, Ultrasonic cutting for surgical applications. Elsevier Ltd., 2015.
[26] M. E. Schafer, Ultrasonic surgical devices and procedures. Elsevier Ltd., 2015.
[27] Stryker, “SONOPET,” 2020. https://neurosurgical.stryker.com/products/sonopet-ultrasonic-aspirator/(accessed Apr. 28, 2020).
[28] Soring, “Ultrasonic assisted bone intrument.” https://www.soering.de/en/products/bone-instrument/(accessed Apr. 28, 2020).
[29] IDOM, “Surgical Ultrasonic Bone-Cutting Device.” https://www.idom.com/project/surgical-ultrasonic-bone-cutting-device/(accessed Apr. 28, 2020).
[30] A. MacBeath, “Ultrasonic Bone Cutting,” University of Glasgow, 2006.
[31] S. Cochran, “Piezoelectricity and basic configurations for piezoelectric ultrasonic transducers,” in Ultrasonic Transducers: Materials and Design for Sensors, Actuators and Medical Applications, Woodhead Publishing Limited, 2012, pp. 3-35.
[32] T. Consulting, “Ultrasonic Vessel Sealing and Dissection Devices,” 2017. https://www.youtube.com/watch?v=dROH9no9yME (accessed May 5, 2020).
[33] Ethicon, “Ethicon Product Catalog by Category.” https://www.ethicon.com/na/system/files?file=2020-03/035213-191202-Ethicon-Product-Catalog-Submission-File-v3.pdf (accessed Apr. 24, 2020).
[34] S. D. McCarus and L. K. S. Parnell, “The Origin and Evolution of the HARMONIC® Scalpel,” Gynecol. Surg. Technol. Int., vol. 35.
[35] B. J. O'Daly, E. Morris, G. P. Gavin, J. M. O'Byrne, and G. B. McGuinness, “High-power low-frequency ultrasound: A review of tissue dissection and ablation in medicine and surgery,” J. Mater. Process. Technol., vol. 200, no. 1-3, pp. 38-58, 2008, doi: 10.1016/j.jmatprotec.2007.11.041.
[36] J. Li, H. Liu, J. Li, Y. Yang, and S. Wang, “Piezoelectric transducer design for an ultrasonic scalpel with enhanced dexterity for minimally invasive surgical robots,” Proc. Inst. Mech. Eng. Part C J. Mech. Eng. Sci., vol. 234, no. 7, pp. 1271-1285, 2020, doi: 10.1177/0954406219892750.
[37] S. Sherrit et al., “Novel horn designs for ultrasonic/sonic cleaning, welding, soldering, cutting, and drilling,” Smart Struct. Mater. 2002 Smart Struct. Integr. Syst., vol. 4701, no. May, pp. 353-360, 2002, doi: 10.1117/12.474671.
[38] S. A. Askins, M. A. Us, and B. Dolgin, “Folded_horn_patent_US20040047485A1 (Done 1652020),” vol. 1, no. 19, 2004.
[39] A. Feeney and M. Lucas, “A Comparison of Two Configurations for a Dual-Resonance Cymbal Transducer,” IEEE Trans. Ultrason. Ferroelectr. Freq. Control, vol. 65, no. 3, pp. 489-496, 2018, doi: 10.1109/TUFFC.2018.2793310.
[40] A. Feeney, “Nitinol Cymbal Transducers for Tuneable Ultrasonic Devices,” 2014, [Online]. Available: http://theses.gla.ac.uk/% 5Cnhttp://theses.gla.ac.uk/5805/.
[41] F. Bejarano, A. Feeney, and M. Lucas, “A cymbal transducer for power ultrasonics applications,” Sensors Actuators, A Phys., vol. 210, pp. 182-189, 2014, doi: 10.1016/j.sna.2014.02.024.
[42] R. Lockhart et al., “Silicon micromachined ultrasonic scalpel for the dissection and coagulation of tissue,” Biomed. Microdevices, vol. 17, no. 4, pp. 1-12, 2015, doi: 10.1007/s10544-015-9981-6.
[43] A. Lal and R. M. White, “Silicon microfabricated horns for power ultrasonics,” Int. Conf. Solid-State Sensors Actuators, Eurosensors IX, Proc., vol. 1, pp. 405-408, 1995, doi: 10.1109/sensor.1995.717221.
[44] T. Jiang, C. Xia, S. Cochran, and Z. Huang, “Improved performance of d31-mode needle-actuating transducer with PMN-PT piezocrystal,” IEEE Trans. Ultrason. Ferroelectr. Freq. Control, vol. 65, no. 8, pp. 1415-1422, 2018, doi: 10.1109/TUFFC.2018.2838566.
[45] N. Velotti et al., “Comparison between LigaSure and Harmonic in Laparoscopic Sleeve Gastrectomy: A Single-Center Experience on 422 Patients,” vol. 2019, 2019.
[46] H. Krentel, H. C. Verhoeven, L. A. Torres-de, and R. L. De Wilde, “Laparoscopic ultrasonic dissectors: technology update by a review of literature,” pp. 1-7, 2019.
[47] B. Person, Æ. D. A. Vivas, Æ. D. Ruiz, Æ. M. Talcott, J. E. Coad, and Æ. S. D. Wexner, “Comparison of four energy-based vascular sealing and cutting instruments: A porcine model,” pp. 534-538, 2008, doi: 10.1007/s00464-007-9619-8.
[48] K. J. Wikiel, T. N. Robinson, and E. L. Jones, “Energy in robotic surgery,” no. November 2019, pp. 1-9, 2020, doi: 10.21037/ales.2020.03.06.
[49] S. C. Yang, J. Ahn, J. H. Kim, J. W. Yi, M. H. Hur, and K. Lee, “Comparison of the vessel sealer Extend® with harmonic ACE® in robotic bilateral axillary-breast approach thyroid surgery,” vol. 9, no. 2, pp. 164-171, 2020, doi: 10.21037/gs.2020.01.18.
[50] X. Yu, J. Zhou, H. Liang, Z. Jiang, and L. Wu, “Progress in Materials Science Mechanical metamaterials associated with stiffness, rigidity and compressibility: A brief review,” Prog. Mater. Sci., vol. 94, pp. 114-173, 2018, doi: 10.1016/j.pmatsci.2017.12.003.
[51] STEMINC, Steiner & Martins, Inc. “Bolt Clamped Langevin Transducer 40 KHz SMBLTD45F40H.” [Online] Available: https://www.steminc.com/PZT/fr/bolt-clamped-langevin-tranducer-40-khz
[52] MEGGITT, Ferroperm™ Piezoceramics, “Ferroperm™ data sheets from Meggitt Denmark.” [Online] Available: https://www.meggittferroperm.com/resources/data-sheets/

1. An ultrasonic transducer for surgical applications, the ultrasonic transducer comprising:
a back mass;
a front mass;
an ultrasonic actuator arrangement held between the back mass and the front mass;
an ultrasonic horn arrangement forward of the front mass,
wherein the back mass, ultrasonic actuator arrangement, front mass and ultrasonic horn arrangement are arranged along a longitudinal axis of the transducer,
wherein vibrations generated by the ultrasonic actuator arrangement are conducted into the front mass and into the ultrasonic horn arrangement along a vibrational energy transfer path and are amplitude amplified by the ultrasonic horn arrangement,
and wherein one or more of the back mass, front mass and ultrasonic horn arrangement includes a plurality of openings opening towards the longitudinal axis and intersecting the vibrational energy transfer path and configured to provide an increased mechanical compliance in a direction along the vibrational energy transfer path.

2. The ultrasonic transducer according to claim 1 wherein the transducer is a Langevin transducer.

3. The ultrasonic transducer according to claim 1 wherein the vibrational energy transfer path in the back mass, front mass and/or ultrasonic horn includes an annular portion, the plurality of openings intersecting the vibrational energy transfer path being provided through a wall of the annular portion.

4. The ultrasonic transducer according to claim 1, wherein each opening has a substantially uniform cross section along its depth.

5. The ultrasonic transducer according to claim 1 wherein the openings each have the same size.

6. The ultrasonic transducer according to claim 1 wherein the openings are arranged based on a repeating pattern.

7. The ultrasonic transducer according to claim 1 wherein there are three or more openings.

8. The ultrasonic transducer according to claim 1 wherein the length of the transducer, measured from the proximal end of the back mass to the distal end of the horn, along the vibrational energy transfer path, is not more than 40 mm.

9. The ultrasonic transducer according to claim 8 wherein the maximum diameter of the transducer, measured in a direction perpendicular to the length, is not more than 15 mm.

10. The ultrasonic transducer according to claim 1 wherein in operation, the plurality of openings provide substantially no longitudinal to torsional mode conversion.

11. The ultrasonic transducer according to claim 1 wherein the openings are provided in an achiral array.

12. The ultrasonic transducer according to claim 1 wherein the plurality of openings is not provided in the ultrasonic horn arrangement.

13. The ultrasonic transducer according to claim 1 wherein the openings are provided in a reflective symmetrical array.

14. The ultrasonic transducer according to claim 1 wherein the openings are arranged based on a repeating pattern.

15. The ultrasonic transducer according to claim 1 wherein the openings are arranged substantially randomly, or offset randomly from a notional regular repeating pattern.

16. The ultrasonic transducer according to claim 1 wherein there are 20 or more openings.

17. The ultrasonic transducer according to claim 1 wherein, for a planar cross section taken perpendicular to the longitudinal axis at a position along the longitudinal axis coinciding with a maximum total number of openings intersecting the plane, the openings occupy at least 10% of the circumference of the front mass or back mass.

18. The ultrasonic transducer according to claim 1 wherein the front mass comprises a proximal portion in contact with the ultrasonic actuator arrangement and a distal portion connected to the ultrasonic horn arrangement and an intermediate portion disposed between the proximal portion and the distal portion, and wherein the openings are provided in the intermediate portion.

19. A surgical tool comprising an ultrasonic transducer comprising:
a back mass;
a front mass;
an ultrasonic actuator arrangement held between the back mass and the front mass;
an ultrasonic horn arrangement forward of the front mass,
wherein the back mass, ultrasonic actuator arrangement, front mass and ultrasonic horn arrangement are arranged along a longitudinal axis of the transducer,
wherein vibrations generated by the ultrasonic actuator arrangement are conducted into the front mass and into the ultrasonic horn arrangement along a vibrational energy transfer path and are amplitude amplified by the ultrasonic horn arrangement,
and wherein one or more of the back mass, front mass and ultrasonic horn arrangement includes a plurality of openings opening towards the longitudinal axis and intersecting the vibrational energy transfer path and configured to provide an increased mechanical compliance in a direction along the vibrational energy transfer path.

20. A method of operation of an ultrasonic transducer, the ultrasonic transducer comprising:
a back mass;
a front mass;
an ultrasonic actuator arrangement held between the back mass and the front mass;
an ultrasonic horn arrangement forward of the front mass,
wherein the back mass, ultrasonic actuator arrangement, front mass and ultrasonic horn arrangement are arranged along a longitudinal axis of the transducer,
wherein vibrations generated by the ultrasonic actuator arrangement are conducted into the front mass and into the ultrasonic horn arrangement along a vibrational energy transfer path and are amplitude amplified by the ultrasonic horn arrangement,
and wherein one or more of the back mass, front mass and ultrasonic horn arrangement includes a plurality of openings opening towards the longitudinal axis and intersecting the vibrational energy transfer path and configured to provide an increased mechanical compliance in a direction along the vibrational energy transfer path.

21. The method according to claim 20, wherein the ultrasonic transducer is operated at a power in the range 1-1000 W.
